(19) United States
(12) Patent Application Publication
(10) Pub. No.: US 2003/0104445 A1
(43) Pub. Date: Jun. 5, 2003

(54) RNA dependent RNA polymerase mediated protein evolution

(76) Inventors: Robert J. Hayes, Pasadena, CA (US); Anna-Marie Aguinaldo, Altadena, CA (US)

Correspondence Address: Robin M. Silva, Dorsey & Whitney LLP, Intellectual Property Department, Four Embarcadero Center, Suite 300, San Francisco, CA, 94111-4187, US

(21) Appl. No.: 10/256,281

(22) Filed: Sep. 25, 2002

Related U.S. Application Data
(60) Provisional application No. 60/325,113, filed on Sep. 25, 2001.

(51) Int. Cl. C12Q 1/68; G01N 33/53; C07H 21/04; C12P 19/34; C12N 9/00; C12P 21/02; C12N 5/06; C07K 14/435

(52) U.S. Cl. 435/6; 435/7.1; 435/69.1; 435/320.1; 435/183; 435/325; 435/91.2; 530/350; 536/23.2

(57) ABSTRACT

The invention relates to the use of RNA dependent RNA polymerase to generate libraries of proteins, and to methods of making and methods and compositions utilizing the libraries.

### CROSS REFERENCE TO RELATED APPLICATIONS

This application claims the benefit of the filing date of Ser. No. 60/325,113, filed on Sep. 25, 2001 under 35 U.S.C. § 119(e).

### FIELD OF THE INVENTION

The invention relates to the use of RNA dependent RNA polymerase to generate libraries of proteins, and to methods of making and methods and compositions utilizing the libraries.

### BACKGROUND OF THE INVENTION

Proteins and enzymes with novel functions and properties may be created using a variety of different methods. Current methods include random techniques, such as directed molecular evolution and random mutagenesis, as well as rational design approaches. Approaches using directed molecular evolution start with a known natural protein, utilize several rounds of mutagenesis, functional screening, and/or selection and propagation to identify candidate sequences encoding proteins with novel functions. The advantage of this process is that it may be used to rapidly evolve any protein without knowledge of its structure. Several different mutagenesis strategies exist, including point mutagenesis by error-prone PCR, cassette mutagenesis, and DNA shuffling (see for example Stemmer, et al. (1994) Nature 370:389-391; Stemmer, et al., (1994) Proc. Natl. Acad. Sci. USA, 91:10747-10751; U.S. Pat. Nos. 5,603,793, 5,830721, 5,811,238, and U.S. Pat. No. 6,426,224).

Computational methods provide a comprehensive rational design approach to generating novel proteins and enzymes. There are a wide variety of methods known for generating and evaluating sequences. These include, but are not limited to, sequence profiling (Bowie and Eisenberg, Science 253(5016): 164-70, (1991)), rotamer library selections (Dahiyat and Mayo, Protein Sci 5(5): 895-903 (1996); Dahiyat and Mayo, Science 278(5335): 82-7 (1997); Desjarlais and Handel, Protein Science 4: 2006-2018 (1995); Harbury et al, PNAS USA 92(18): 8408-8412 (1995); Kono et al., Proteins: Structure, Function and Genetics 19: 244-255 (1994); Hellinga and Richards, PNAS USA 91: 5803-5807 (1994)); and residue pair potentials (Jones, Protein Science 3: 567-574, (1994)). (see Altschul and Koonin, Trends Biochem Sci 23(11): 444-447. (1998); (see Altschul et al., J. Mol. Biol. 215(3): 403 (1990) and Lockless and Ranganathan, Science 286:295-299 (1999), Pattern discovery in Biomolecular Data: Tools, Techniques, and Applications; edited by Jason T. L. Wang, Bruce A. Shapiro, Dennis Shasha. New York: Oxford University, 1999.)

RNA viruses and retroviruses exhibit enormous genetic variability. An individual RNA virus or retro virus does not form a homogeneous population but rather a set of viral variants. Both replication and recombination contribute to the generation of viral variants. RNA viruses replicate with an intrinsic replication error some 300 times greater than DNA-based microbes and approximately 10⁶ times greater than eukaryotic genomes. This is the consequence of a total lack of replication proofreading machinery and results in an intrinsic nucleotide substitution error of approximately 0.05-1 nucleotide mutations per genome per cycle (Angel, et al. (1994) Proc. Natl. Acad. Sci. USA, 91: 11787-11791).

RNA-RNA recombination is responsible for even more profound changes within the viral genome (see Figlerowicz & Bibillo (2000) RNA, 6: 339-351 and references cited therein). Studies conducted over the last decade clearly indicate that the exchange of RNA genetic information can occur between viral strains, viral species, or viral and cellular RNAs. In addition to having a role in the evolution of the viral RNA genome and in the generation of new viral strains, RNA recombination can correct errors that arise during RNA replication (Figlerowicz, et al. (1998) J. Virology, 72: 9192-9200).

Accordingly it is an object of the present invention to utilize the replication error rate and promiscuous recombination associated with viral RNA-dependent RNA polymerases to rapidly generate proteins and enzymes with novel characteristics.

### SUMMARY OF THE INVENTION

The present invention provides methods for generating protein libraries comprising providing at least a first positive template RNA comprising a 3′ RNA-dependent RNA polymerase (RdRp) recognition signal and a target gene. An RdRp enzyme and NTPs are added to generate a plurality of negative recombinant nucleic acids, followed by the addition of a reverse transcriptase (RT) enzyme and dNTPs to generate a plurality of positive recombinant nucleic acid strands. The positive recombinant nucleic acid strands are amplified, incorporated into expression vectors, and the expression vectors transformed into suitable host cells. Additional steps comprise screening the transformed host cells for a desired phenotype and isolating variant proteins.

In an additional object, the invention provides methods for generating protein libraries comprising providing at least a first positive template RNA comprising a 3′ RdRp recognition signal, a 5′ RdRp recognition signal and a target gene. An RdRp enzyme and NTPs are added to generate a plurality of negative recombinant nucleic acids, followed by the addition of an RT enzyme and dNTPs to generate a plurality of positive recombinant nucleic acid strands. The positive recombinant nucleic acid strands are amplified, incorporated into expression vectors, and the expression vectors transformed into suitable host cells. Additional steps comprise screening the transformed host cells for a desired phenotype and isolating variant proteins.

In an further object, the invention provides methods for generating protein libraries comprising providing a plurality of first positive template RNAs each comprising a different target gene, adding a RT and dNTPs to generate a plurality of negative and positive variant DNA recombinant strands. The negative and positive variant DNA recombinant strands are amplified, incorporated into expression vectors, and the expression vectors transformed into a plurality of suitable host cells. Additional steps comprise screening the transformed host cells for a desired phenotype and isolating variant proteins.

In an further object, the invention provides methods for generating protein libraries comprising providing at least one DNA template comprising a T7 promoter and a target gene, adding a RT and dNTPs to generate a plurality of negative and positive variant DNA recombinant strands. The negative and positive variant DNA recombinant strands are amplified, incorporated into expression vectors, and the expression vectors transformed into a plurality of suitable host cells. Additional steps comprise screening the transformed host cells for a desired phenotype and isolating variant proteins.

In an further object, the invention provides methods for generating protein libraries comprising providing a host cell expressing an RdRp, introducing at least a first template RNA comprising a 3′ RdRp recognition signal, a 5″ RdRp recognition signal, and a target gene, generating a plurality of host cells containing different variant protein sequences, and screening the host cells for a desired phenotype. Additional steps comprise isolating variant proteins.

In an further object, the invention provides methods for generating protein libraries comprising providing a host cell expressing an RdRp, introducing at least a first template RNA comprising a 3′ RdRp recognition signal, a 5″ RdRp recognition signal, and a target gene, generating a plurality of host cells containing different variant nucleic acid sequences, amplifying the variant nucleic acid sequences, incorporating the sequences into expression vectors, transforming a plurality of suitable host cells, and screening the host cells for a desired phenotype. Additional steps comprise isolating variant proteins.

Additional objects comprise synthesizing a plurality of recombinant amplicons and experimentally or computationally recombining the recombinant amplicons to generate secondary libraries comprising variant sequences.

Target genes may be naturally occurring genes, designed genes, homologous or non-homologous gens.

RdRps may be naturally occurring or variant RdRps.

### BRIEF DESCRIPTION OF THE DRAWINGS

FIG. 1 depicts template switching mediated by RNA dependent RNA polymerase.

FIG. 2 depicts template switching mediated by MLV reverse transcriptase between two genes encoding beta-lactamase genes.

FIG. 3 schematic of the vector used to generate RNA templates for template switching mediated by reverse transcriptase.

FIG. 4 depicts the beta-lactamase donor and acceptor templates for template switching mediated by reverse transcriptase.

FIGS. 5A and 5B are schematics of the dehalogenase constructs. The amino acids in HD5C that differ from HDWT are indicated in italicized lettering below the solid vertical lines. The restriction sites found in HD5C that are not present in HDWT are indicated by the dotted vertical lines (except NdeI and Not1).

FIG. 6 depicts the crossover regions for dehalogenase recombination mediated by reverse transcriptase.

FIG. 7 depicts the sequencing results from various dehalogenase recombinants (Example 2).

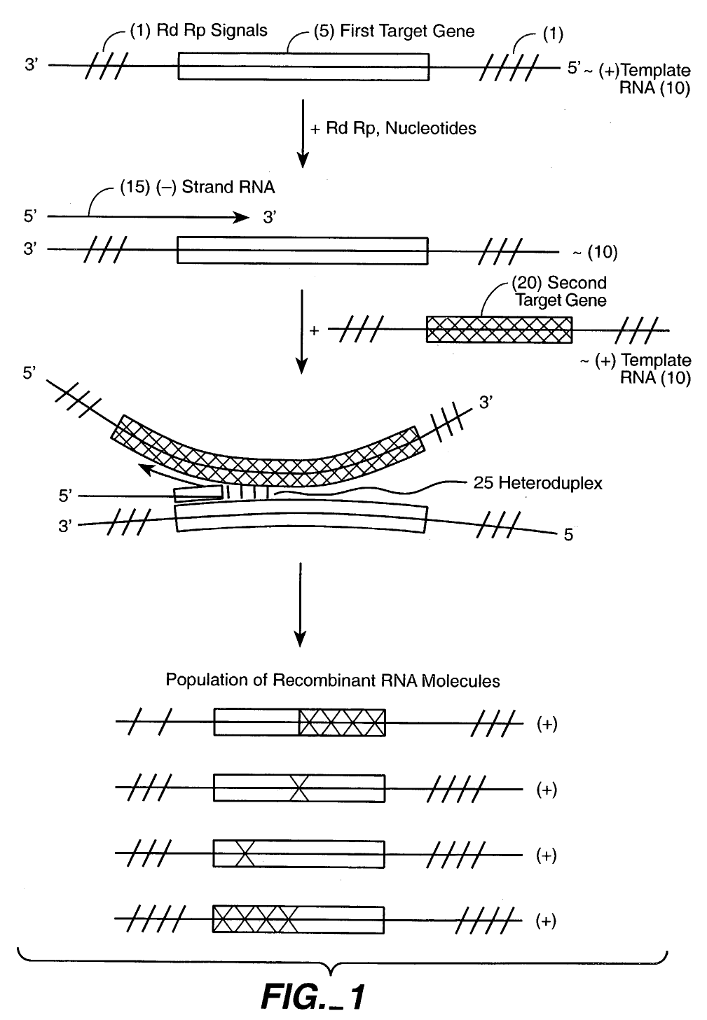

### DETAILED DESCRIPTION OF THE INVENTION

The present invention is directed to methods of generating protein libraries using a combination of experimental and computational methods which rely on the infidelity of RNA enzymes. That is, many RNA enzymes will “switch” strands, allowing a recombination of sorts, and additionally, many enzymes involved in RNA synthesis are error prone, thus allowing the introduction of random mutations. As described herein, a wide variety of methods may be used to generate the vector, cellular and protein libraries of the present invention. Generally, three basic steps are involved. The first step is a generation (generating) step involving the generation of one or more nucleic acid templates. The second step is a recombination (shuffling) step, in which an enzyme that uses RNA as a template (i.e., RNA-dependent RNA polymerase, reverse transcriptase, RNA polymerase) is used to mediate recombination of one or more nucleic acid sequences. The third step is a library (making) step, which generally includes the generation of expression vector, cellular libraries, and protein libraries.

Overview of RNA Shuffling

In the methods used herein, recombination is generally mediated using either RNA-dependent RNA polymerase (RdRp) or reverse transcriptase (RT). Most RNA viruses use RdRp and RT enzymes to replicate their genomes. Some RNA viruses, however, use host-encoded RNA polymerases (Chang & Taylor, (2002) EMBO, 21:157-164). Collectively, these enzymes have minimal proof-reading activities and consequently their error rates are about ten thousand times higher than those encountered during DNA replication. This means that the genome of any individual RNA virus particle will contain an average of one or more mutations from the consensus wild-type sequence for that virus species (Ball, (2001) “Replication Strategies of RNA Viruses”, in Fields Virology, Vol 1, 4^{th} ed., pp 105-118).

RNA-dependent RNA polymerases (RdRps) and reverse transcriptases (RTs) also mediate recombination. Two RNA recombination mechanisms have been proposed: breakage-rejoining and copy-choice. The breakage-rejoining mechanism takes place with the splicing of group 11 introns and can result in the production of some recombinant RNAs by the Qβ replicase (Kim & Kao, (2001) Proc. Natl. Acad. Sci. USA, 98:4972-4977). Several lines of evidence suggest that RNA viruses and retroviruses recombine according to a copy choice mechanism. The copy choice mechanism assumes that recombinants are formed when the viral replication complex changes RNA templates during nascent RNA- or DNA-strand synthesis (template switching event). Recombination can occur between homologous RNA molecules and non-homologous RNA molecules (Figlerowicz & Bibillo, (2000) RNA, 6:339-351).

RNA-dependent RNA polymerase is used by many RNA viruses to replicate their genome. In single-stranded positive RNA viruses, replication catalyzed by RdRp takes place in two stages: 1) synthesis of a complementary (negative-strand) RNA using the virus genomic RNA as a template; and, 2) synthesis of progeny virus genomic RNA using the negative-strand RNA as a template (Hayes & Buck, (1990) Cell, 63:363-368).

Three basic models have been proposed for the replication of positive-stranded RNA viruses, which involve intermediates with different structures (Buck (1996) Adv. Virus Res., 47: 159-251). In model I, the RdRp recognizes a promoter at the 3′ end of the positive-strand RNA template and starts to synthesize a complementary negative-strand. The nascent negative-strand remains base-paired to the positive-strand in the region where the polymerase binds to the template and is actively synthesizing RNA (i.e., generating a heteroduplex structure). The 5′ tail of nascent strand is not base-paired to the template; thus most of the replicative intermediate is in a single-stranded form. Continuation of the reaction leads to the formation of a free negative-strand product and releases the positive-strand template. The polymerase then recognizes a promoter at the 3′ end of the negative-strand and using the negative-strand as a template, starts to synthesize a progeny positive-strand, giving a second type of replicative intermediate. As before, the nascent strand is only base-paired to the template in the region of the active site of the polymerase where RNA synthesis is taking place, so that this replicative intermediate is also mainly single-stranded. Before the synthesis of the first progeny positive-strand has been completed, initiation of synthesis of further positive-strands takes place, giving a replicative intermediate consisting of a full-length negative-stranded template, to which are attached several nascent positive-strands which again are largely in a single-stranded form. The process continues to synthesize and release multiple copies of the progeny positive-stranded RNA (Buck (1996) Adv. Virus Res., 47: 159-251).

The first stage of model II is essentially the same as that of model 1, except that the negative-strand formed remains base-paired with the positive-stranded template, giving a replicative intermediate consisting of partially double-stranded and partially single-stranded structure. The reaction continues to give a fully double-stranded RNA replicative form. In this model, no free negative-strand is synthesized. The polymerase then recognizes a promoter at the end of the replicative form dsRNA containing the 3′ end of the negative-strand and the 5′ end of the positive-strand. Synthesis of progeny positive-stranded RNA commences using the negative-strand as a template by a strand-displacement mechanism, giving rise to replicative intermediates consisting of double-stranded RNA with one, or following reinitiations, several single-stranded 5′ tails of the full length positive-strands. The first full-length positive-strand to be released from the replicative intermediate will be the original template strand; continued reaction will ten result in the synthesis and release of multiple progeny positive-strands (Buck (1996) Adv. Virus Res., 47: 159-251).

The formation of the double-stranded replicative form in model III is exactly the same as in model II. However, synthesis of progeny positive-stranded RNA using the negative-strand of the dsRNA only displaces the positive-strand of the dsRNA transiently in the region where RNA synthesis is taking place. The replicative intermediates formed consist of double-stranded RNA with one or several single-stranded tails, but unlike the replicative intermediate in model 11 in which the single-stranded tails are the displaced 5′tails of full-length positive-strands, these single-stranded tails belong to the nascent, incomplete progeny positive-strands (Buck (1996) Adv. Virus Res., 47: 159-251).

Specificity in template selection and the stringency of promoter recognition vary among the RdRps. In addition, other structural features (i.e. 5′ cap structure) and cis-acting sequences are necessary for replicase assembly, promoter recognition, and initiation of negative-strand synthesis. In particular, cis-acting sequences have been found to effect the efficiency of replication in vivo, the rate of replication, the ability of the viral RNA to act as a template, and the frequency of aberrant replication (Buck, (1996) Adv. Virus Res., 47: 159-251). Elements that affect positive and negative-strand synthesis include CAA repeats, hairpin motifs, tRNA acceptor arms, tRNA anticodon arms, pseudoknot and/or stem/loop structures, and bulge sequences (Rajendran, et al. (2002) J. Virol., 76: 1707-1717; Osman, et al., (2000) J. Virology, 74: 11671-11680). Some of these cis-acting sequences, i.e. CCA repeats, can be recognized by several RdRps (Rajendran, et al. (2002) J. Virol., 76: 1707-1717).

RNA recombination depends on RNA replication. The prevailing model for RNA recombination posits that recombination occurs when the RNA replicase switches strands during RNA synthesis (i.e., copy choice model; Figlerowicz, et al. (1998) J. Virol., 72: 9192-9200). Depending on the primary structure of the recombining molecules and on the location of the recombinant junction sites, three types of RNA recombination can be distinguished: homologous, aberrant homologous and non-homologous (Figlerowicz, (2000) Nucleic Acids Res., 28: 1714-1723). Generally, homologous recombination occurs between identical or highly homologous sequences. Nucleotide identity as short as 15 nucleotides will support homologous recombination (Nagy & Bujarski (1995) J. Virol., 69: 131-140).

Local hybridization between RNAs (i.e., RNA-RNA heteroduplex formation) and a hairpin structure efficiently promotes non-homologous RNA recombination (Figlerowicz, et al. (1998) J. Virol., 72: 9192-9200). The proposed model for non-homologous recombination in brome mosaic virus (BMV) assumes that recombinants are formed during the synthesis of minus RNA strands. Viral replicase initiates at the 3′ end of the donor RNA strand (positive-strand) and then the enzyme switches to the acceptor RNA strand (negative-strand) within the heterduplex structure (Figlerowicz, et al. (1998) J. Virol., 72: 9192-9200).

Reverse transcriptase (RT) is used by retroviruses to ensure that two copies of their single-stranded genomic RNA are present in each viral particle. RTs exhibit low template affinity and processivity, consequently, retroviral populations exhibit high levels of variation, allowing the virus to escape host immune systems and acquire resistance to antiretroviral drugs. The rate of genetic variation depends on the mutation and recombination rates per replication cycle (Hwang, et al. (2001) Proc. Natl. Acad. Sci. USA, 98: 12209-12214).

When a retrovirus enters the host cell, reverse transcriptase (RT) converts the genomic RNA into a double-stranded DNA that integrates into the host's genome. During synthesis of the first DNA strand, the reverse transcriptase can switch template from one to the other copy of the genomic RNA, a phenomenon known as “copy-choice”. Template-switching events may result in deletions, deletions with insertions, insertions, duplications, homology or non-homologous recombination. Thus, the high rate of recombination in retroviruses is the result of frequent template switching occurring during reverse transcription. Recent evidence suggests that specific RNA structures are involved in triggering the switch (Negroni & Buc, (2001) Nature Reviews, 2: 151-158).

Approach to Library Generation using Enzymes that use RNA as a Substrate

Accordingly, the present invention provides methods for generating libraries by providing a nucleic acid template that can be recombined and/or mutated by an RNA enzyme. Once made, the libraries may be additionally manipulated either experimentally or computationally to create new libraries that may be screened and experimentally tested.

In a preferred embodiment, the libraries are generated by recombination (i.e. shuffling). “Recombination” or “shuffling” or “promiscuous recombination” as used herein means recombination of one or more protein, DNA or RNA sequences. Recombination may be done experimentally and/or computationally (e.g. “in silico shuffling”). See for example, U.S. Pat. No. 6,319,714; WO 00/42559 WO 00/42560; and WO 00/42561; all of which are hereby incorporated by reference in their entirety. As will be appreciated by those of skill in the art, other means of generating libraries, i.e. such as computational methods, may also be used.

By “libraries” herein is meant a collection of nucleic acid sequences, amino acid sequences, cells, or vectors. Thus, libraries generated by the methods of the present invention may be expression vector libraries, cellular libraries, or nucleic acid or protein libraries. By “expression vector libraries” herein is meant a plurality of expression vectors wherein generally each vector within the library contains at least one member of the library. Generally, members of expression vector libraries are nucleic acid sequences. Ideally each vector contains a single and different library member, although as will be appreciated by those in the art, some vectors within the library may not contain a library member and some may contain more than one member. Suitable vectors are described below.

By a “cellular library” herein is meant a plurality of cells wherein generally each cell within the library contains at least one member of the library. Ideally each cell contains a single and different library member, although as will be appreciated by those in the art, some cells within the library may not contain a library member and some may contain more than one library member. When methods other than retroviral infection are used to introduce the library members into a plurality of cells, the distribution of library members within the individual cell members of the cellular library may vary widely, as it is generally difficult to control the number of nucleic acids which enter a cell during electroporation and other transformation methods. Suitable cell types for cellular libraries are described below. In addition, as will be appreciated by those in the art, a cellular library generally includes a single cell type, although in some embodiments, a cellular library may contain two or more cell types.

By “nucleic acid libraries” herein is meant a collection of nucleic acid sequences, preferably, but not always, recombinant nucleic acid sequences. By “recombinant nucleic acid sequences” herein is meant “non-naturally occurring” or “synthetic” or “recombinant” or grammatical equivalents thereof, herein is meant a nucleic acid sequence that is not found in nature; that is, the nucleotide sequence usually has been intentionally modified. In the context of this invention, “recombinant nucleic acid sequences include nucleic acid sequences generated by template switching events mediated by RdRps or RT or by the introduction of errors (e.g. mutants). As will be appreciated by those of skill in the art, recombinant nucleic acid sequences may contain point mutations introduced during replication, as well deletions, deletions with insertions, insertions, duplications, homologous and/or non-homologous recombination introduced during template switching.

By “protein libraries” herein is meant a collection of amino acid sequences, preferably, but not always variant amino acid sequences. By “variant amino acid sequence” herein is meant a protein sequence that differs from another protein sequence. Preferably, a variant protein sequence has at least one amino acid that differs from the amino acid defined by the target amino acid sequence. As outlined below, this target amino acid sequence may be a wild-type sequence or a variant sequence.

Nucleic Acid Templates

In a preferred embodiment, the libraries of the present invention are generated by shuffling nucleic acid templates. By “nucleic acid template” herein is meant a single or double stranded nucleic acid.

By “nucleic acid” or “oligonucleotide” or grammatical equivalents herein means at least-two nucleotides covalently linked together. A nucleic acid of the present invention will generally contain phosphodiester bonds, although some cases, as outlined below, nucleic acid analogs are included that may have alternate backbones, comprising, for example, phosphoramide (Beaucage et al., Tetrahedron 49(10):1925 1993) and references therein; Letsinger, J. Org. Chem. 35:3800 (1970); Sprinzl et al., Eur. J. Biochem. 81:579 (1977); Letsinger et al., Nucl. Acids Res. 14:3487 (1986); Sawai et al, Chem. Lett. 805 (1984), Letsinger et al., J. Am. Chem. Soc. 110:4470 (1988); and Pauwels et al., Chemica Scripta 26:141 (1986)), phosphorothioate (Mag et al., Nucleic Acids Res. 19:1437 (1991); and U.S. Pat. No. 5,644,048), phosphorodithioate (Briu et al., J. Am. Chem. Soc. 111:2321 (1989), O-methylphophoroamidite linkages (see Eckstein, Oligonucleotides and Analogues: A Practical approach, Oxford University Press), and peptide nucleic acid backbones and linkages (see Egholm, J. Am. Chem. Soc. 114:1895 (1992); Meier et al., Chem. Int. Ed. Engl. 31:1008 (1992); Nielsen, Nature, 365:566 (1993); Carlsson et al., Nature 380:207 (1996), all of which are incorporated by reference). Other analog nucleic acids include those with positive backbones (Denpcy et al., Proc. Natl. Acad. Sci. U SA 92:6097 (1995); non-ionic backbones (U.S. Pat. Nos. 5,386,023, 5,637,684, 5,602,240, 5,216,141 and 4,469,863; Kiedrowshi et al., Angew. Chem. Intl. Ed. English 30:423 (1991); Letsinger et al., J. Am. Chem. Soc. 110:4470 (1988); Letsinger et al., Nucleoside & Nucleotide 13:1597 (1994); Chapters 2 and 3, ASC Symposium Series 580, “Carbohydrate Modifications in Antisense Research”, Ed. Y. S. Sanghui and P. Dan Cook; Mesmaeker et al., Bioorganic & Medicinal Chem. Lett. 4:395 (1994); Jeffs et al., J. Biomolecular NMR 34:17 (1994); Tetrahedron Lett. 37:743 (1996)) and non-ribose backbones, including those described in U.S. Pat. Nos. 5,235,033 and 5,034,506, and Chapters 6 and 7, ASC Symposium Series 580, “Carbohydrate Modifications in Antisense Research”, Ed. Y. S. Sanghui and P. Dan Cook. Nucleic acids containing one or more carbocyclic sugars are also included within the definition of nucleic acids (see Jenkins et al., Chem. Soc. Rev. (1995) pp 169-176). Several nucleic acid analogs are described in Rawls, C & E News Jun. 2, 1997 page 35. All of these references are hereby expressly incorporated by reference. These modifications of the ribose-phosphate backbone may be done to facilitate the addition of additional moieties such as labels, or to increase the stability and half-life of such molecules in physiological environments.

As will be appreciated by those in the art, all of these nucleic acid analogs may find use in the present invention. In addition, mixtures of naturally occurring nucleic acids and analogs may be made. Alternatively, mixtures of different nucleic acid analogs, and mixtures of naturally occurring nucleic acids and analogs may be made.

The nucleic acids may be single stranded or double stranded, as specified, or contain portions of both double stranded or single stranded sequence. The nucleic acid may be DNA, both genomic and cDNA, RNA or a hybrid, where the nucleic acid contains any combination of deoxyribo- and ribonucleotides, and any combination of bases, including uracil, adenine, thymine, cytosine, guanine, inosine, xathanine hypoxathanine, isocytosine, isoguanine, etc. As used herein, the term “nucleoside” includes nucleotides and nucleoside and nucleotide analogs, and modified nucleosides such as amino modified nucleosides. In addition, “nucleoside” includes non-naturally occurring analog structures. Thus for example the individual units of a peptide nucleic acid, each containing a base, are referred to herein as a nucleoside backbone to increase stability and half life of such molecules in physiological environments. In the context of the present invention, preferred nucleic acids are RNA molecules, including both positive and negative-strands.

As will be appreciated by those in the art, the depiction of a single strand (“Watson”) also defines the sequence of the other strand (“Crick”). A nucleic acid template may comprise an intact gene, or a fragment of a gene encoding functional domains of a protein, such as enzymatic domains, regulatory sequences, binding domains, etc., as well as smaller gene fragments. The template nucleic acid may be from any organism, either prokaryotic or eukaryotic. The template sequence may be naturally occurring, a variant, a product of a computational step, etc. As used herein, “Watson” will refer to the positive (e.g. sense) strand of a nucleic acid (e.g. RNA), and “Crick” will refer to the negative (e.g., antisense) strand.

RNA Templates

In a preferred embodiment, the libraries are generated using an RNA template. By “RNA template” herein is meant a ribonucleic acid sequence comprising a positive-sense template ribonucleic acid (RNA). By “positive template RNA” herein is meant a single-stranded messenger-sense or “Watson” RNA molecule. In alternative embodiments, negative template or “Crick” RNA molecules may be used.

In a preferred embodiment, the libraries are generated using an RNA template comprising a positive template ribonucleic acid, recognition signals for an RdRp, and at least one target gene.

By “recognition signals” herein is meant nucleotide sequences required for RNA replication. Preferably, the nucleotide sequences are cis-acting nucleotide sequences and include promoters for negative- and positive-strand RNA synthesis. Depending on the virus, additional sequences may also be required for efficient replication. These additional sequence may be located 1) at the 3′-termini of positive-strands; 2) at the 5′-terminal regions of positive-strands and 3′-terminal regions of negative-strands. These sequences can be considered together, as mutations that affect one will necessarily affect the other and complementary secondary structures can sometimes be formed for both termini; and, 3) internal sequences (Buck, (1996) Adv. Virus Res., 47: 159-251).

For example, without being bound by theory, a number of 3′-terminal cis-acting sequences appear to effect the efficiency of RNA replication, template switching and/or error production. There are a variety of 3′ terminal cis-acting sequences including, but not limited to, sequences that can be folded into tRNA-like structures found in several genera of plant viruses in the alpha-like virus supergroup (Buck (1996) Adv. Virus Res., 47:159-251). Other 3′-termini cis-acting sequences include hairpin motifs, stem-loop structures, pseudoknots, bulge sequences, poly(A) tails, and CCC/CCA repeats (Rajendran, et al. (2002) J. Virol., 76: 1707-1717; Osman, et al., (2000) J. Virology, 74: 11671-11680; Buck (1996) Adv. Virus Res., 47:159-251).

In addition, there are 5′-terminal sequences in a variety of viruses that effect the efficiency of viral RNA replication. For example, cloverleaf structures have been identified in poliovirus. The 5′ untranslated regions of brome mosaic virus RNAs resemble consensus sequences for the internal control regions (ICR1 and ICR2) of tRNA promoters. In the mature tRNA, ICR1 and ICR2 correspond to the D-loop and T-loop respectively. ICR-like sequences have been found in other bromoviruses, cucumoviruses, tobamoviruses, tobraviruses, tymoviruses, and tobacco necrosis satellite virus. In brome mosaic virus (BMV), it has been proposed that the 5′-terminal regions of BMV RNA 2 can be folded into a stem-loop structure with the ICR2-like motif in the loop and the ICR1-like motif comprising part of the stem. Similar stem-loop structures were predicted for the 5′ termini of BMV RNAs 1 and 3, RNAs of cucumber mosaic virus, cowpea chlorotic mottle virus, and alfalfa mosaic virus. Moreover, more than one stem-loop structure may be required. For example, the 5′ untranslated region of beet necrotic yellow vein virus can be folded into a structure containing several stem-loop structures. Other 5′ terminal elements include multiple CAA repeats (Buck (1996) Adv. Virus Res., 47:159-251). Accordingly, suitable 5′ sequences include, but are not limited to, sequences that fold into stem-loop structures, and CAA repeats.

Internal cis-acting elements, in either intercistronic or coding regions that contribute to efficient RNA replication have been identified in a number of virus RNAs. In some cases, such sequences may be useful to maintain an optimal RNA structure for binding of the replicase complex to promoters at the termini of the positive- or negative-stranded RNAs, or to promote processivity of the replicase during RNA synthesis. In other cases, it is possible that the replicase could bind to internal sequences for a particular purpose, e.g., translation repression, or for an obligatory step in the assembly or modification of RNA complexes. For example, a sequence of about 150 nucleotides in the 5′ region of the 244 nucleotide intercistronic region of BMV RNA 3, contains ICR-like motifs (Buck (1996) Adv. Virus Res., 47:159-251). Accordingly, suitable internal sequences include, but are not limited to ICR-like motifs.

In a preferred embodiment, the RNA template comprises a positive template RNA and a target gene.

In a preferred embodiment, the RNA template comprises a positive template RNA, a 3′ promoter for negative-strand synthesis, and a target gene.

In a preferred embodiment, the RNA template comprises a positive template RNA, promoters for negative- and positive-strand synthesis and a target gene.

In a preferred embodiment, the RNA template comprises a positive template RNA, promoters for negative- and positive-strand synthesis, a 3′-terminal cis-acting sequence, and a target gene. Terminal 3′ cis-acting sequences are selected from the group consisting of tRNA like structures, hairpin motifs, stem-loop structures, pseudoknots, bulge sequences, poly(A) tails, and CCC/CCA repeats.

In a preferred embodiment, the RNA template comprises a positive template RNA, promoters for negative- and positive-strand synthesis, a 3′-terminal cis-acting sequence, a 5′-terminal cis-acting sequence and a target gene. Terminal 3′ cis-acting sequences are selected from the group consisting of tRNA like structures, hairpin motifs, stem-loop structures, pseudoknots, bulge sequences, poly(A) tails, and CCC/CCA repeats. Terminal 5′ cis-acting sequences are selected from the group consisting of cloverleaf structures and ICR-like stem-loop structures.

DNA Templates

In a preferred embodiment, the libraries are generated using a DNA template. By “DNA template” herein is meant a deoxyribonucleic acid sequence comprising an RNA polymerase promoter and a target gene. Preferably, the RNA polymerase promoter is the T7 promoter, although as will be appreciated by those of skill in the art, other RNA polymerase promoters may be used including the T5, T3 and SP6 promoters. Additionally, the DNA templates may comprise selectable markers, labels, etc., described below.

In a preferred embodiment, the DNA template comprises a T7 promoter, a target gene, and sequence elements encoding promoters for negative- and positive-strand synthesis of RNA templates transcribed from the DNA template, a 3′-terminal cis-acting sequence, a 5′-terminal cis-acting sequence. Terminal 3′ cis-acting sequences are selected from the group consisting of tRNA like structures, hairpin motifs, stem-loop structures, pseudoknots, bulge sequences, poly(A) tails, and CCC/CCA repeats.

In a preferred embodiment, the DNA template comprises a T7 promoter and a target gene.

In a preferred embodiment, the DNA template comprises a T7 promoter, a 3′ promoter for negative-strand synthesis of RNA, and a target gene.

In a preferred embodiment, the DNA template comprises a T7 promoter, promoters for negative- and positive-strand synthesis of RNA and a target gene.

In a preferred embodiment, the DNA template comprises a T7 promoter, promoters for negative- and positive-strand synthesis of RNA, a 3′-terminal cis-acting sequence, and a target gene. Terminal 3′ cis-acting sequences are selected from the group consisting of tRNA like structures, hairpin motifs, stem-loop structures, pseudoknots, bulge sequences, poly(A) tails, and CCC/CCA repeats.

In a preferred embodiment, the DNA template comprises a T7 promoter, promoters for negative- and positive-strand synthesis of RNA, a 3′-terminal cis-acting sequence, a 5′-terminal cis-acting sequence and a target gene. Terminal 3′ cis-acting sequences are selected from the group consisting of tRNA like structures, hairpin motifs, stem-loop structures, pseudoknots, bulge sequences, poly(A) tails, and CCC/CCA repeats. Terminal 5′ cis-acting sequences are selected from the group consisting of cloverleaf structures and ICR-like stem-loop structures.

In a preferred embodiment, the DNA template comprises a RNA polymerase promoter and a target gene.

Target Genes

By “target gene” herein is meant a gene encoding a target protein for which a library of variant protein sequences is desired. As will be appreciated by those of skill in the art, any number of target proteins find use in the invention. By “target protein” herein is meant at least two covalently attached amino acids, which includes proteins, polypeptides, oligopeptides and peptides. The protein may be made up of naturally occurring amino acids and peptide bonds, or in some special embodiments, synthetic peptidomimetic structures, i.e., “analogs” such as peptoids [see Simon et al., Proc. Natl. Acad. Sci. U.S.A. 89(20:9367-71 (1992)], generally depending on the method of synthesis. Thus “amino acid”, or “peptide residue”, as used herein means both naturally occurring and synthetic amino acids. For example, homo-phenylalanine, citrulline, and noreleucine are considered amino acids for the purposes of the invention. “Amino acid” also includes imino acid residues such as proline and hydroxyproline. In addition, any amino acid representing a component of the variant proteins of the present invention can be replaced by the same amino acid but of the opposite chirality. Thus, any amino acid naturally occurring in the L-configuration (which may also be referred to as the R or S, depending upon the structure of the chemical entity) may be replaced with an amino acid of the same chemical structural type, but of the opposite chirality, generally referred to as the D-amino acid but which can additionally be referred to as the R- or the S-, depending upon its composition and chemical configuration. Such derivatives generally have the property of greatly increased stability, and therefore are advantageous in the formulation of compounds which may have longer in vivo half lives, when administered by oral, intravenous, intramuscular, intraperitoneal, topical, rectal, intraocular, or other routes.

In the preferred embodiment, the amino acids are in the S- or L-configuration. If non-naturally occurring side chains are used, non-amino acid substituents may be used, for example to prevent or retard in vivo degradations. Proteins including non-naturally occurring amino acids may be synthesized or in some cases, made recombinantly; see van Hest et al., FEBS Lett 428:(1-2) 68-70 May 22 1998 and Tang et al., Abstr. Pap Am. Chem. S218: U138 Part 2 Aug. 22, 1999, both of which are expressly incorporated by reference herein.

Aromatic amino acids may be replaced with D- or L-naphylalanine, D- or L-phenylglycine, D- or L-2-thieneylalanine, D- or L-1-, 2-, 3- or 4-pyreneylalanine, D- or L-3-thieneylalanine, D- or L-(2-pyridinyl)-alanine, D- or L-(3-pyridinyl)-alanine, D- or L-(2-pyrazinyl)-alanine, D- or L-(4-isopropyl)-phenylglycine, D-(trifluoromethyl)-phenylglycine, D-(trifluoromethyl)-phenylalanine, D-p-fluorophenylalanine, D- or L-p-biphenylphenylalanine, D- or L-p-methoxybiphenylphenylalanine, D- or L-2-indole(alkyl)alanines, and D- or L-alkylalanines where alkyl may be substituted or unsubstituted methyl, ethyl, propyl, hexyl, butyl, pentyl, isopropyl, iso-butyl, sec-isotyl, iso-pentyl, and non-acidic amino acids of C1-C20. Acidic amino acids can be substituted with non-carboxylate amino acids while maintaining a negative charge, and derivatives or analogs thereof, such as the non-limiting examples of (phosphono)alanine, glycine, leucine, isoleucine, threonine, or serine; or sulfated (e.g., —SO₃H) threonine, serine, or tyrosine.

Other substitutions may include nonnatural hydroxylated amino acids may made by combining “alkyl” with any natural amino acid. The term “alkyl” as used herein refers to a branched or unbranched saturated hydrocarbon group of 1 to 24 carbon atoms, such as methyl, ethyl, n-propyl, isoptopyl, n-butyl, isobutyl, t-butyl, octyl, decyl, tetradecyl, hexadecyl, eicosyl, tetracisyl and the like. Alkyl includes heteroalkyl, with atoms of nitrogen, oxygen and sulfur. Preferred alkyl groups herein contain 1 to 12 carbon atoms. Basic amino acids may be substituted with alkyl groups at any position of the naturally occurring amino acids lysine, arginine, ornithine, citrulline, or (guanidino)-acetic acid, or other (guanidino)alkyl-acetic acids, where “alkyl” is define as above. Nitrile derivatives (e.g., containing the CN-moiety in place of COOH) may also be substituted for asparagine or glutamine, and methionine sulfoxide may be substituted for methionine. Methods of preparation of such peptide derivatives are well known to one skilled in the art.

In addition, any amide linkage in any of the variant polypeptides may be replaced by a ketomethylene moiety. Such derivatives are expected to have the property of increased stability to degradation by enzymes, and therefore possess advantages for the formulation of compounds which may have increased in vivo half lives, as administered by oral, intravenous, intramuscular, intraperitoneal, topical, rectal, intraocular, or other routes.

Additional amino acid modifications of amino acids of variant polypeptides of to the present invention may include the following: Cysteinyl residues may be reacted with alpha-haloacetates (and corresponding amines), such as 2-chloroacetic acid or chloroacetamide, to give carboxymethyl or carboxyamidomethyl derivatives. Cysteinyl residues may also be derivatized by reaction with compounds such as bromotrifluoroacetone, alpha-bromo-beta-(5-imidozoyl)propionic acid, chloroacetyl phosphate, N-alkylmaleimides, 3-nitro-2-pyridyl disulfide, methyl 2-pyridyl disulfide, p-chloromercuribenzoate, 2-chloromercuri-4-nitrophenol, or chloro-7-nitrobenzo-2-oxa-1,3-diazole. Histidyl residues may be derivatized by reaction with compounds such as diethylprocarbonate e.g., at pH 5.5-7.0 because this agent is relatively specific for the histidyl side chain, and para-bromophenacyl bromide may also be used; e.g., where the reaction is preferably performed in 0.1M sodium cacodylate at pH 6.0.

Lysinyl and amino terminal residues may be reacted with compounds such as succinic or other carboxylic acid anhydrides. Derivatization with these agents is expected to have the effect of reversing the charge of the lysinyl residues.

Other suitable reagents for derivatizing alpha-amino-containing residues include compounds such as imidoesters, e.g., as methyl picolinimidate; pyridoxal phosphate; pyridoxal; chloroborohydride; trinitrobenzenesulfonic acid; O-methylisourea; 2,4 pentanedione; and transaminase-catalyzed reaction with glyoxylate. Arginyl residues may be modified by reaction with one or several conventional reagents, among them phenylglyoxal, 2,3-butanedione, 1,2-cyclohexanedione, and ninhydrin according to known method steps. Derivatization of arginine residues requires that the reaction be performed in alkaline conditions because of the high pKa of the guanidine functional group. Furthermore, these reagents may react with the groups of lysine as well as the arginine epsilon-amino group. The specific modification of tyrosyl residues per se is well known, such as for introducing spectral labels into tyrosyl residues by reaction with aromatic diazonium compounds or tetranitromethane.

N-acetylimidizol and tetranitromethane may be used to form O-acetyl tyrosyl species and 3-nitro derivatives, respectively. Carboxyl side groups (aspartyl or glutamyl) may be selectively modified by reaction with carbodiimides (R′-N-C-N-R′) such as 1-cyclohexyl-3-(2-morpholinyl-(4-ethyl) carbodiimide or 1-ethyl-3-(4-azonia-4,4-dimethylpentyl) carbodiimide. Furthermore aspartyl and glutamyl residues may be converted to asparaginyl and glutaminyl residues by reaction with ammonium ions.

Glutaminyl and asparaginyl residues may be frequently deamidated to the corresponding glutamyl and aspartyl residues. Alternatively, these residues may be deamidated under mildly acidic conditions. Either form of these residues falls within the scope of the present invention.

The target proteins of the present invention may be from prokaryotes and eukaryotes, such as bacteria (including extremeophiles such as the archebacteria), fungi, insects, fish, and mammals. Suitable mammals include, but are not limited to, rodents (rats, mice, hamsters, guinea pigs, etc.), primates, farm animals (including sheep, goats, pigs, cows, horses, etc) and in the most preferred embodiment, from humans.

Thus, by “target protein” herein is meant a protein for which a library of variants is desired. As will be appreciated by those in the art, any number of target proteins will find use in the present invention. Specifically included within the definition of “protein” are fragments and domains of known proteins, including functional domains such as enzymatic domains, binding domains, etc., and smaller fragments, such as turns, loops, etc. That is, portions of proteins may be used as well. In addition, “protein” as used herein includes proteins, oligopeptides and peptides. In addition, protein variants, i.e. non-naturally occurring protein analog structures, may be used.

Suitable target proteins include, but are not limited to, industrial and pharmaceutical proteins, including ligands, cell surface receptors, antigens, antibodies, cytokines, hormones, transcription factors, signaling modules, cytoskeletal proteins and enzymes.

Specifically, preferred target proteins include, but are not limited to, those with known or predictable structures (including variants):

1) cytokines (IL-1ra (+receptor complex), IL-1 (receptor alone), IL-1a, IL-1b (including variants and or receptor complex), IL-2, IL-3, IL-4, IL-5, IL-6, IL-8, IL-10, IFN-β, INF-γ, IFN-α-2a; IFN-α-2B, TNF-α; CD40 ligand (chk), Human Obesity Protein Leptin, Granulocyte Colony-Stimulating Factor, Bone Morphogenetic Protein-7, Ciliary Neurotrophic Factor, Granulocyte-Macrophage Colony-Stimulating Factor, Monocyte Chemoattractant Protein 1, Macrophage Migration Inhibitory Factor, Human Glycosylation-Inhibiting Factor, Human Rantes, Human Macrophage Inflammatory Protein 1 Beta, human growth hormone, Leukemia Inhibitory Factor, Human Melanoma Growth Stimulatory Activity, neutrophil activating peptide-2, Cc-Chemokine Mcp-3, Platelet Factor M2, Neutrophil Activating Peptide 2, Eotaxin, Stromal Cell-Derived Factor-1, Insulin, Insulin-like Growth Factor I, Insulin-like Growth Factor II, Transforming Growth Factor B1, Transforming Growth Factor B2, Transforming Growth Factor B3, Transforming Growth Factor A, Vascular Endothelial growth factor (VEGF), acidic Fibroblast growth factor, basic Fibroblast growth factor, Endothelial growth factor, Nerve growth factor, Brain Derived Neurotrophic Factor, Ciliary Neurotrophic Factor, Platelet Derived Growth Factor, Human Hepatocyte Growth Factor, Fibroblast Growth Factor (including but not limited to alternative splice variants, abundant variants, and the like), Glial Cell-Derived Neurotrophic Factor, and haemopoietic receptor cytokines (including but not limited to erythropoietin, thrombopoietin, and prolactin), APM1 (including, but not limited to adipose most abundant gene transript 1), and the like;

2) other extracellular signaling moieties, including, but not limited to, Sonic hedgehog, protein hormones such as chorionic gonadotrophin and leutenizing hormone;

3) blood clotting and coagulation factors including, but not limited to, TPA and Factor VIIa; coagulation factor IX; coagulation factor X; PROTEIN S protein; Fibrinogen and Thrombin; ANTITHROMBIN III; streptokinase and urokinase, retevase, and the like;

4) transcription factors and other DNA binding proteins, including but not limited to, histones, p53; myc; PIT1; NFkB;AP1 ;JUN; KD domain, homeodomain, heat shock transcription factors, stat, zinc finger proteins (e.g. zif268);

5) antibodies, antigens, and trojan horse antigens, including, but not limited to, immunoglobulin super family proteins, including but not limited to CD4 and CD8, Fc receptors, T-cell receptors, MHC-I, MHC-II, CD3, and the like. Also, immunoglobulin-like proteins, including but not limited to fibronectin, pkd domain, integrin domains, cadhrin, invasins, cell surface receptors with Ig-like domains, and the like. Intrabodies, and the like; Anti-Her/2 neu antibody (e.g. Herceptin); Anti-VEGF; Anti-CD20 (Rituxan), among others;

6) intracellular signaling modules, including, but not limited to, kinases, phosphatases, G-proteins Phosphatidylinositol 3-kinase (PI3-kinase) kinase, Phosphatidylinositol 4-kinase, wnt family members including but not limited to wnt-1 through wnt 15, EF hand proteins including calmodulin, troponin C, S100B, calbindin and D9k; NOTCH; MEK; MAPK; ubitquitin and ubiquitin like proteins, including UBL1, UBL5, UBL3 and UBL4, and the like;

7) viral proteins, including, but not limited to, hemagglutinin trimerization domain and HIV Gp41 ectodomain (fusion domain); viral coat proteins, viral receptors, integrases, proteases, reverse transcriptases;

8) receptors, including, but not limited to, the extracellular region of human tissue factor cytokine-binding region Of Gp130, G-CSF receptor, erythropoietin receptor, Fibroblast Growth Factor receptor, TNF receptor, IL-1 receptor, IL-1 receptor/IL1ra complex, IL-4 receptor, INF-γ receptor alpha chain, MHC Class I, MHC Class II, T Cell Receptor, Insulin receptor, insulin receptor tyrosine kinase and human growth hormone receptor; Lectins; GPCRs, including but not limited to G-Protein coupled receptors; ABC Transporters/Multidrug resistance proteins; Na and K channels; Nuclear Hormone Receptors; Aquaporins; Transporters, RAGE (receptor for advanced glycan end points), TRK -A, -B, -C, and the like, and haemopoietic receptors;

9) enzymes including, but not limited to, hydrolases such as proteases/proteinases, synthases/synthetases/ligases, decarboxylases/lyases, peroxidases, ATPases, carbohydrases, lipases; isomerases such as racemases, epimerases, tautomerases, or mutases; transferases, hydrolases, kinases, reductases/oxidoreductases, hydrogenases, polymerases, phophatases, and proteasomes anti-proteasomes, (e.g., MLN341). Suitable enzymes include, but limited to, those listed in the Swiss-Prot enzyme database;

10) additional proteins including but not limited to heat shock proteins, ribosomal proteins, glycoproteins, motor proteins, transporters, drug resistance proteins, kinetoplasts and chaperonins;

11) antimicrobial peptides;

12) small proteins including but not limited to metal ligand and disulfide-bridged proteins such as metallothionein, Kunitiz-type inhibitors, crambin, snake and scorpion toxins, and trefoil proteins; antimicrobial peptides such as defensins, thoredoixn, fereodoxin, transferetin, and the like;

13) protein domains and motifs including, but not limited to, SH-2 domains, SH-3 domains, Pleckstrin homology domains, WW domains, SAM domains, kinase domains, death domains, RING finger domains, Kringle domains, heparin-binding domains, cysteine-rich domains, leucine zipper domains, zinc finger domains, nucleotide binding motifs, transmembrane helices, and helix-turn-helix motifs. Additionally, ATP/GTP-binding site motif A; Ankyrin repeats; fibronectin domain; Frizzled (fz) domain; GTPase binding domain; C-type lectin domain; PDZ domain; ‘Homeobox’ domain; Kruieppel-associated box (KRAB); Leucine zipper; DEAD and DEAH box families; ATP-dependent helicases; HMG½ signature; DNA mismatch repair proteins mutL/hexB/PMS1 signature; Thioredoxin family active site; Thioredoxins; Annexins repeated domain signature; Clathrin light chains signatures; Myotoxins signature; Staphylococcal enterotoxins/Streptococcal pyrogenic exotoxins signatures; Serpins signature; Cysteine proteases inhibitors signature; Chaperonins; Heat shock; WD domains; EGF-like domains; Immunoglobulin domains, Immunoglobulin-like proteins and the like;

14) specific protein sites or other subsets of residues, including but not limited to protease cleavage/recognition sites, phosphorylation sites, metal binding sites, and signal sequences. Additionally, proteins having post-translational modifications include, but are not limited to: N-glycosylation site; O-glycosylation site; Glycosaminoglycan attachment site; Tyrosine sulfation site; cAMP- and cGMP; dependent protein kinase phosphorylation site; Protein kinase C phosphorylation site; Casein kinase II phosphorylation site; Tyrosine kinase phosphorylation site; N-myristoylation site; Amidation site; Aspartic acid and asparagine hydroxylation site; Vitamin K-dependent carboxylation domain; Phosphopantetheine attachment site; Prokaryotic membrane lipoprotein lipid attachment site; Prokaryotic N-terminal methylation site; Prenyl group binding site (CAAX box); Intein N- and C-terminal splicing motif profiles, and the like;

15) proteins involved in motility, including but not limited to chemokines, S100 family proteins (including but not limited to ENRAGE);

16) peptides—defensins;

17) peptide ligands including, but not limited to, a short region from the HIV-1 envelope cytoplasmic domain (shown to block the action of cellular calmodulin), regions of the Fas cytoplasmic domain (death-inducing apoptotic or G protein inducing functions), magainin, a natural peptide derived from Xenopus (anti-tumor and anti-microbial activity), short peptide fragments of a protein kinase C isozyme, βPKC (blocks nuclear translocation of full-length βPKC in Xenopus oocytes following stimulation), SH-3 target peptides, naturitic peptides (AMP, BMP, and CMP), and fibrinopeptides and neuropeptides;

18) presentation scaffolds or “ministructures” including, but are not limited to, minibody structures (see for example Bianchi et al., J. Mol. Biol. 236(2):649-59 (1994), and references cited therein, all of which are incorporated by reference), maquettes (Grosset et al. Biochemistry 40:5474-5487 (2001)), loops on beta-sheet turns and coiled-coil stem structures (see, for example, Myszka et al., Biochem. 33:2362-2373 (1994) and Martin et al., EMBO J. 13(22):5303-5309 (1994), incorporated by reference), zinc-finger domains, transglutaminase linked structures, cyclic peptides, B-loop structures, coiled coils, helical bundles, helical hairpins, and beta hairpins; and,

19) ion channel protein domains, including but not limited to sodium, calcium, potassium, and chloride, including their component subunit. Examples of extracellular ligand-gated ion channels include nAChR receptors, GABA and glycine, 5H-T, MOD-1, P(2×), glutamate, NMDA, AMPA, Kainate receptors, GluR-B, ORCC, P2×3, Inward rectifying channels, ROMK, IRK, BIR, and the like. Examples of voltage-gated ion channels, Examples of intracellular ligand-gated ion channels, Mechanosensative and cell volume-regulated ion channels, and the like.

In addition, a preferred embodiment utilizes target proteins such as random peptides. That is, there is a significant amount of work being done in the area of utilizing random peptides in high throughput screening techniques to identify biologically relevant (particularly disease states) proteins. The peptides are randomized, either fully randomized or they are biased in their randomization, e.g. in nucleotide/residue frequency generally or per position. By “randomized” or grammatical equivalents herein is meant that each nucleic acid and peptide consists of essentially random nucleotides and amino acids, respectively. Thus, any amino acid residue may be incorporated at any position. The synthetic process can be designed to generate randomized peptides and/or nucleic acids, to allow the formation of all or most of the possible combinations over the length of the nucleic acid, thus forming a library of randomized nucleic acids. See also U.S. Ser. No. 10/218,102, incorporated herein by reference in its entirety.

In a preferred embodiment, the target protein is a variant protein, including, but not limited to, mutant proteins comprising one or a plurality of substitutions, insertions or deletions, including chimeric genes, and genes that have been optimized in any number of ways, including experimentally or computationally.

In a preferred embodiment, the target protein is a chimeric protein. A chimeric protein (sometimes referred to as a “fusion protein”) in this context means a protein that has sequences from at least two different sequences operably linked or fused. The chimeric protein may be made using either a single linkage point or a plurality of linkage points. In addition, the source of the parent protein sequences may be as listed above for scaffold proteins, e.g. prokaryotes, eukaryotes, including archebacteria and viruses, etc.

As will be appreciated by those in the art, chimeric proteins may be made from different naturally occurring proteins in a gene family (e.g. one with recognizable sequence or structural homology) or by artificially joining two or more distinct genes. For example, the binding domain of a human protein may be fused with the activation domain of a mouse gene, etc.

The sequence of the chimeric gene may be been constructed synthetically (e.g. arbitrary or targeted portions of two or more genes are crossed over randomly or purposely), experimentally (e.g. through homologous recombination or shuffling techniques) or computationally (e.g. using genetic annealing programs, “in silico shuffling”, alignment programs, etc.). For the purposes of the invention, these techniques can be done at the protein or nucleic acid level.

In a preferred embodiment, the target protein is actually a product of a computational design cycle and/or screening process. That is, a first round of the methods of the invention may produce one or more sequences for which further analysis is desired.

Although several classes of proteins have been stated herein, this should not be construed as an exhaustive list, but rather some examples of proteins that may be optimized using the experimental and computational methodologies outlined herein.

In a preferred embodiment, recombination using RNA enzymes is done using at least one target gene.

In a preferred embodiment, more than one target gene is recombined. That is, the target gene may be an ensemble or set of structures such as those represented by a set of homologous sequences. Homologous in this context means that two or more sequences are capable of being recombined using the techniques of the invention.

As is known in the art, a number of different programs may be used to identify whether or not a protein (or nucleic acid as discussed below) has sequence identity or similarity to a known sequence. Sequence identity and/or similarity is determined using standard techniques known in the art, including, but not limited to, the local sequence identity algorithm of Smith & Waterman, Adv. Appl. Math., 2:482 (1981), by the sequence identity alignment algorithm of Needleman & Wunsch, J. Mol. Biol., 48:443 (1970), by the search for similarity method of Pearson & Lipman, Proc. Natl. Acad. Sci. U.S.A., 85:2444 (1988), by computerized implementations of these algorithms (GAP, BESTFIT, FASTA, and TFASTA in the Wisconsin Genetics Software Package, Genetics Computer Group, 575 Science Drive, Madison, Wis.), the Best Fit sequence program described by Devereux et al., Nucl. Acid Res., 12:387-395 (1984), preferably using the default settings, or by inspection. Preferably, percent identity is calculated by FastDB based upon the following parameters: mismatch penalty of 1; gap penalty of 1; gap size penalty of 0.33; and joining penalty of 30, “Current Methods in Sequence Comparison and Analysis,” Macromolecule Sequencing and Synthesis, Selected Methods and Applications, pp 127-149 (1988), Alan R. Liss, Inc. All references cited in this paragraph are incorporated by reference in their entirety.

An example of a useful algorithm is PILEUP. PILEUP creates a multiple sequence alignment from a group of related sequences using progressive, pairwise alignments. It can also plot a tree showing the clustering relationships used to create the alignment. PILEUP uses a simplification of the progressive alignment method of Feng & Doolittle, J. Mol. Evol. 35:351-360 (1987); the method is similar to that described by Higgins & Sharp CABIOS 5:151-153 (1989). Useful PILEUP parameters including a default gap weight of 3.00, a default gap length weight of 0.10, and weighted end gaps. Another example of a useful algorithm is the BLAST algorithm, described in: Altschul et al., J. Mol. Biol. 215, 403-410, (1990); Altschul et al., Nucleic Acids Res. 25:3389-3402 (1997); and Karlin et al., Proc. Natl. Acad. Sci. U.S.A. 90:5873-5787 (1993). A particularly useful BLAST program is the WU-BLAST-2 program which was obtained from Altschul et al., Methods in Enzymology, 266:460-480 (1996); http://blast.wustl/edu/blast/README.html]. WU-BLAST-2 uses several search parameters, most of which are set to the default values. The adjustable parameters are set with the following values: overlap span=1, overlap fraction=0.125, word threshold (T)=11. The HSP S and HSP S2 parameters are dynamic values and are established by the program itself depending upon the composition of the particular sequence and composition of the particular database against which the sequence of interest is being searched; however, the values may be adjusted to increase sensitivity.

An additional useful algorithm is gapped BLAST as reported by Altschul et al., Nucl. Acids Res., 25:3389-3402. Gapped BLAST uses BLOSUM-62 substitution scores; threshold T parameter set to 9; the two-hit method to trigger ungapped extensions; charges gap lengths of k a cost of 10+k; Xu set to 16, and Xg set to 40 for database search stage and to 67 for the output stage of the algorithms. Gapped alignments are triggered by a score corresponding to ˜22 bits.

A “%” amino acid sequence identity value is determined by the number of matching identical residues divided by the total number of residues of the “longer” sequence in the aligned region. The “longer” sequence is the one having the most actual residues in the aligned region (gaps introduced by WU-Blast-2 to maximize the alignment score are ignored).

In a similar manner, “percent (%) nucleic acid sequence identity” with respect to the coding sequence of the polypeptides identified herein is defined as the percentage of nucleotide residues in a candidate sequence that are identical with the nucleotide residues in the coding sequence of the target protein. A preferred method utilizes the BLASTN module of WU-BLAST-2 set to the default parameters, with overlap span and overlap fraction set to 1 and 0.125, respectively.

The alignment may include the introduction of gaps in the sequences to be aligned. In addition, for sequences which contain either more or fewer amino acids than the target protein, it is understood that in one embodiment, the percentage of sequence identity will be determined based on the number of identical amino acids in relation to the total number of amino acids. In percent identity calculations relative weight is not assigned to various manifestations of sequence variation, such as, insertions, deletions, substitutions, etc.

In one embodiment, only identities are scored positively (+1) and all forms of sequence variation including gaps are assigned a value of “0”, which obviates the need for a weighted scale or parameters as described below for sequence similarity calculations. Percent sequence identity can be calculated, for example, by dividing the number of matching identical residues by the total number of residues of the “shorter” sequence in the aligned region and multiplying by 100. The “longer” sequence is the one having the most actual residues in the aligned region.

Other useful ensembles include sets of related proteins, sets of related structures, artificial created ensembles, etc.

RNA-dependent RNA polymerases (RdRps)

In a preferred embodiment, the methods of the invention involve starting with a RNA template and using an RdRp to generate a plurality of primary variant recombinant nucleic acid sequences. By “RdRp” herein is meant RNA-dependent RNA polymerase. RdRps may be naturally occurring or recombinant.

In a preferred embodiment, naturally occurring RdRps are used. By “naturally occurring” or “wild type” or grammatical equivalents, herein is meant an RdRp that is found in nature and includes allelic variations; that is, the amino acid sequence or a nucleotide sequence encoding the RdRp has not been intentionally modified. Accordingly, by “non-naturally occurring” or “synthetic” or “recombinant” or grammatical equivalents thereof, herein is meant an RdRp that is not found in nature; that is, the amino acid sequence or a nucleotide sequence encoding the RdRp usually has been intentionally modified.

Naturally occurring RdRps may be purified from single-stranded or double-stranded RNA viruses as described in Hayes & Buck (1990) Cell, 63: 363-368; Rajendran, et al. (2002) J. Virology, 76: 1707-17117; Osman &Buck, (1996) J. Virology, 70: 6227-6234; Hayes, et al., (1992) J. Gen. Virology, 73:1597-1600; and Galarza, et al. (1996) J. Virology, 70: 2360-2368; all of which are hereby incorporated in their entirety by reference. Suitable virus supergroups for the purification of RdRp include Picrona (i.e., polioviruses), Poty (tobacco etch viruse), Sobemo (southern bean mosaic virus), Arteri (avian infectious bronchitis virus), Astro (human astrovirus), phage (phage Qβ), Flavi (yellow fever virus), Pesti (bovine diarrhea virus), Carmo (tomato bushy stunt virus), Tymo ( turnip yellow mosaic virus), Tobamo (brome mosaic virus), and Rubi (sindbis virus) (see Buck (1996) Adv. Virus Res., 47: 159-251; hereby incorporated by reference in its entirety). Although several viral supergroups have been stated herein, this should not be construed as an exhaustive list, but rather some examples of viruses from which purified RdRps may be obtained.

In a preferred embodiment, RdRps are produced recombinantly in bacteria, yeast, fungal, insect or mammalian cells (Kim & Kao, (2001) Proc. Natl. Acad. Sci. USA, 98: 4972-4977); or in in vitro expression systems, such as bacterial cell lysates, rabbit cell lysates, wheat germ cell lysates or plant cell lysates.

In Vitro Recombination Using RdRps

In a preferred embodiment, the present invention provides methods for generating libraries by providing a positive RNA template that may be shuffled, i.e. recombined, in vitro by a viral RdRp. Depending on the RNA template and recognition signals provided, a plurality of negative recombinant nucleic acid strands, or a plurality of negative and positive recombinant nucleic acids strands may be generated. Generally, the positive RNA template is generated from a DNA template. In this embodiment, a target gene is cloned between DNA versions of RdRp recognition sequences. RNA corresponding to the RNA template is transcribed from an upstream RNA polymerase promoter, e.g., T3, T5, T7, or Sp6. The resulting RNA template is purified and used as described below.

In a preferred embodiment, a plurality of negative recombinant nucleic acid strands is generated by in vitro replication using RdRp. In this embodiment, at least one positive template RNA comprising a 3′-RdRp recognition signal and a target gene is added to a reaction mixture comprising an RdRp, and nucleotides and incubated for a time sufficient to generate a population of negative recombinant RNA molecules (see for example Hayes & Buck (1990) Cell, 63: 363-368). A plurality of positive recombinant DNA molecules is generated from the population of negative, recombinant RNA molecules using reverse transcriptase. Nucleic acid amplification is done to generate a population of recombinant nucleic acid amplicons that can then be cloned into an expression vector, and transformed into a suitable host using methods known to those of skill in the art. Suitable amplification methods are known in the art, with PCR being preferred. The resulting cellular library may then be screened for proteins with desired properties either directly or indirectly as described below. In some embodiments, it may be preferable to sequence the clones directly rather than screening for proteins with desired properties. Similarly, the proteins may be purified (for example by using purification or affinity tags) prior to screening.

In a preferred embodiment, a plurality of negative and positive recombinant nucleic acid strands are generated by in vitro replication. In this embodiment, at least a one positive template RNA comprising a 3′ RdRp recognition signal, a 5′ RdRp recognition signal and a target gene is added to a reaction mixture comprising an RdRp and nucleotides and incubated for a time sufficient to generate a population of negative and positive recombinant RNA molecules (see for example Hayes & Buck (1990) Cell, 63: 363-368). A plurality of positive and negative recombinant DNA molecules is generated from the population of negative and positive recombinant RNA molecules using reverse transcriptase. Amplification is used to generate a population of recombinant nucleic acid amplicons that can then be cloned into an expression vector, and transformed into a suitable host using methods known to those of skill in the art. The resulting cellular library may then be screened for proteins with desired properties either directly or indirectly as described below. In some embodiments, it may be preferable to sequence the clones directly rather than screening for proteins with desired properties. Again, purified proteins may be screened as well.

In a preferred embodiment, either the error rate or the rate of recombination may be increased or decreased by: 1) altering the concentration of nucleotides, 2) increasing or decreasing the exent of sequence homology; 3) using modified nucleotides (see Nagy & Bujarski, (1995) J. Virology, 69: 131-140); and, 4) altering the reaction conditions such as the temperature, salt and/or pH.

In some embodiments, RNA chaperones, may be added to 1) effect the rate of recombination; 2) induce recombination; or 3) suppress recombination (Negroni & Buc, (2000) Proc. Natl. Acad. Sci. USA, 97: 6385-6390).

In Vivo Recombination Using RdRps

In a preferred embodiment, the present invention provides methods for generating libraries by providing a positive-strand RNA template that can be recombined, in vivo by a viral RdRp. The methods comprise providing a host cell expressing an RdRp. The gene(s) encoding the RdRp may be stably or transiently integrated into the host cell or expressed from an autonomously replicating plasmid (Price, et al. (2002) J. Virology, 76: 1610-1616; incorporated herein by reference in its entirety). Suitable host cells expression systems are discussed below.

Preferably, a target gene is inserted into a cloning vector between DNA versions of the RdRp recognition sequences and located behind a constitutive or inducible promoter. The vector containing the target gene is then introduced into a host cell via transfection in a stably integrated form or as part of an autonomously replicating vector. Transcription of the DNA template into an RNA template will initiate replication of the RNA template. In some embodiments, RT-PCR and suitable primers are used to reverse transcribe and amplify the population of recombinant RNA sequences. Once amplified, the resultant recombinant DNA sequences may be cloned into an expression vector and sequenced or transformed into a suitable host (discussed below). Alternatively, the replicated form of the RNA may be translated directly by the host ribosomes and the expression of proteins with desired properties detected either directly or indirectly in vivo by cell based assays, or in vitro following extraction from the cells.

In Vitro Recombination Using Reverse Transcriptases (RTs)

In a preferred embodiment, the present invention provides methods for generating libraries by providing a positive RNA template that can be shuffled, i.e. recombined, in vitro by a viral reverse transcriptase (RT). Suitable viral reverse transcriptases include, but are not limited to, reverse transcriptases isolated from Moloney murine leukemia virus (MMLV), human immunodeficiency virus (HIV), and Avian myeloblastosis virus (AMV).

Generally, the positive RNA template is generated from a DNA template. In this embodiment, a target gene is inserted into commercially available cloning vectors, such as pETBlue-1 (Novagen) downstream from an RNA polymerase promoter. Other sequences that may be present on the vector, include a plasmid origin of replication, the lacZ gene, and genes encoding a selectable marker, such as a phenotypic marker, as discussed below. RNAs corresponding to the RNA template are transcribed from an upstream RNA polymerase promoter, i.e., T3, T5, T7, or Sp6. The resulting RNA template is purified and used as described below.

In a preferred embodiment, a plurality of positive and negative recombinant DNA strands are generated by in vitro replication using RT. In this embodiment, a plurality of positive template RNAs each comprising a different target gene is added to a reaction mixture comprising an RT and deoxyribonucleotides and incubated for a time sufficient to generate a population of positive and negative recombinant DNA molecules (see Examples). Amplification is used to generate a population of recombinant nucleic acid amplicons that may then be cloned into an expression vector, and transformed into a suitable host using methods known to those of skill in the art. The resulting cellular library may then be screened for proteins with desired properties either directly or indirectly as described below. In some embodiments, it may be preferable to sequence the clones directly rather than screening for proteins with desired properties, or purify the proteins to screen for activity.

In a preferred embodiment, a plurality of positive and negative recombinant DNA strands is generated by in vitro replication using RT. In this embodiment, at least one DNA template comprising an RNA polymerase promoter and a target gene is transcribed in vitro to generate a plurality of positive RNA templates each comprising a different target gene. A RT and deoxyribonucleotides are added to the population RNA templates to generate a plurality of positive and negative recombinant DNA molecules (see Examples). Amplification, using the polymerase chain reaction is used to generate a population of recombinant nucleic acid amplicons that can then be cloned into an expression vector, and transformed into a suitable host using methods known to those of skill in the art. The resulting cellular library can then be screened for proteins with desired properties either directly or indirectly as described below. In some embodiments, it may be preferable to sequence the clones directly rather than screening for proteins with desired properties, or purify the proteins to screen for activity.

In a preferred embodiment, either the error rate or the rate of recombination may be increased or decreased by: 1) altering the concentration of deoxynucleotides; 2) increasing or decreasing the exent of sequence homology; 3) altering the RT concentrations (see Negroni, et al. (1995) Proc. Natl. Acad. Sci “USA, 92: 6971-6975); 4) altering the concentration of RNA templates (see Negroni, et al. (1995) Proc. Natl. Acad. Sci “USA, 92: 6971-6975); and, 5) using modified nucleotides (see Martinez, et al. (1994) Proc. Natl. Acad. Sci. “USA, 91: 11787-11701; 6) altering the reaction conditions such as the temperature, salt and/or pH.

In some embodiments, RNA chaperones, may be added to 1) effect the rate of recombination; 2) induce recombination; or 3) suppress recombination (Negroni & Buc, (2000) Proc. Natl. Acad. Sci. USA, 97: 6385-6390).

As will be appreciated by those of skill in the art, other RNA polymerases may be used to generate a plurality of recombinant nucleic acids for use in the present invention. For example, host-encoded RNA polymerase II may be used to generate recombinant nucleic acid molecules (Chang & Taylor, (2002) EMBO, 21: 157-164).

As will be appreciated by those of skill in the art, other DNA polymerases may be used to generate a plurality of recombinant nucleic acids for use in the present invention. For example, Taq DNA polymerase may be used to generate recombinant nucleic acid molecules (Zaphiropoulos, (1998) NAR, 26: 2843-2848).

Expression Vectors

A variety of expression vectors may be utilized to express the library proteins. The expression vectors are constructed to be compatible with the host cell type. Expression vectors may comprise self-replicating extrachromosomal vectors or vectors which integrate into a host genome. Expression vectors typically comprise a library member, any fusion constructs, control or regulatory sequences, selectable markers, and/or additional elements.

Preferred bacterial expression vectors include but are not limited to pET, pBAD, pBluescript, pUC, pQE, pGEX, pMAL, and the like.

Preferred yeast expression vectors include pPICZ, pPIC3.5K, and pHIL-SI commercially available from Invitrogen.

Expression vectors for the transformation of insect cells, and in particular, baculovirus-based expression vectors, are well known in the art and are described e.g., in O'Reilly et al., Baculovirus Expression Vectors: A Laboratory Manual (New York: Oxford University Press, 1994).

A preferred mammalian expression vector system is a retroviral vector system such as is generally described in Mann et al., Cell, 33:153-9 (1993); Pear et al., Proc. Natl. Acad. Sci. U.S.A., 90(18):8392-6 (1993); Kitamura et al., Proc. Natl. Acad. Sci. U.S.A., 92:9146-56 (1995); Kinsella et al., Human Gene Therapy, 7:1405-13; Hofmann et al.,Proc. Natl. Acad. Sci. U.S.A., 93:5185-90; Choate et al., Human Gene Therapy, 7:2247 (1996); PCT/US97/01019 and PCT/US97/01048, and references cited therein, all of which are hereby expressly incorporated by reference.

Inclusion of Control or Regulatory Sequences

Generally, expression vectors include transcriptional and translational regulatory nucleic acid sequences which are operably linked to the nucleic acid sequence encoding the library protein. Nucleic acid is operably linked when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation However, enhancers do not have to be contiguous.

The transcriptional and translational regulatory nucleic acid sequences will generally be appropriate to the host cell used to express the library protein, as will be appreciated by those in the art. For example, transcriptional and translational regulatory sequences from E. coli are preferably used to express proteins in E. coli. Similarly, transcriptional and translational regulatory nucleic acid sequences from Bacillus are preferably used to express the library protein in Bacillus. Numerous types of appropriate expression vectors, and suitable regulatory sequences are known in the art for a variety of host cells.

Transcriptional and translational regulatory sequences may include, but are not limited to, promoter sequences, ribosomal binding sites, transcriptional start and stop sequences, translational start and stop sequences, and enhancer or activator sequences. In a preferred embodiment, the regulatory sequences comprise a promoter and transcriptional and translational start and stop sequences.

A suitable promoter is any nucleic acid sequence capable of binding RNA polymerase and initiating the downstream (3′) transcription of the coding sequence of library protein into mRNA. Promoter sequences include constitutive and inducible promoter sequences. The promoters may be naturally occurring promoters, hybrid or synthetic promoters. Hybrid promoters, which combine elements of more than one promoter, are also known in the art, and are useful in the present invention.

A suitable bacterial promoter has a transcription initiation region, which is usually placed proximal to the 5′ end of the coding sequence. The transcription initiation region typically includes an RNA polymerase binding site and a transcription initiation site. In E. coli, the ribosome-binding site is called the Shine-Dalgarno (SD) sequence and includes an initiation codon and a sequence 3-9 nucleotides in length located 3-11 nucleotides upstream of the initiation codon. Promoter sequences for metabolic pathway enzymes are commonly utilized. Examples include promoter sequences derived from sugar metabolizing enzymes, such as galactose, lactose and maltose, and sequences derived from biosynthetic enzymes such as tryptophan. Promoters from bacteriophage, such as the T7 promoter, may also be used. In addition, synthetic promoters and hybrid promoters are also useful; for example, the tac promoter is a hybrid of the trp and lac promoter sequences.

Preferred yeast promoter sequences include the inducible GAL1, 10 promoter, the promoters from alcohol dehydrogenase, enolase, glucokinase, glucose-6-phosphate isomerase, glyceraldehyde-3-phosphate-dehydrogenase, hexokinase, phosphofructokinase, 3-phosphoglycerate mutase, pyruvate kinase, and the acid phosphatase gene.

A suitable mammalian promoter will have a transcription initiating region, which is usually placed proximal to the 5′ end of the coding sequence, and a TATA box, usually located 25-30 base pairs upstream of the transcription initiation site. The TATA box is thought to direct RNA polymerase 11 to begin RNA synthesis at the correct site. A mammalian promoter will also contain an upstream promoter element (enhancer element), typically located within 100 to 200 base pairs upstream of the TATA box. Typically, transcription termination and polyadenylation sequences recognized by mammalian cells are regulatory regions located 3′ to the translation stop codon and thus, together with the promoter elements, flank the coding sequence. The 3′ terminus of the mature mRNA is formed by site-specific post-translational cleavage and polyadenylation. Examples of transcription terminator and polyadenylation signals include those derived from SV40. An upstream promoter element determines the rate at which transcription is initiated and can act in either orientation. Of particular use as mammalian promoters are the promoters from mammalian viral genes, since the viral genes are often highly expressed and have a broad host range. Examples include the SV40 early promoter, mouse mammary tumor virus LTR promoter, adenovirus major late promoter, herpes simplex virus promoter, and the CMV promoter.

Inclusion of a Selectable Marker

In addition, in a preferred embodiment, the expression vector contains one or more selectable genes or parts of selectable marker genes to allow the selection of transformed host cells containing the expression vector, and particularly in the case of mammalian cells, ensures the stability of the vector, since cells which do not contain the vector will generally die. Selection genes are well known in the art and will vary with the host cell used. As will be appreciated by those of skill in the art, other DNA polymerases may be used to generate a plurality of recombinant nucleic acids for use in the present invention. For example, Taq DNA polymerase may be used to generate recombinant nucleic acid molecules (Zaphiropoulos, (1998) NAR, 26: 2843-2848).

The bacterial expression vector may also include at least one selectable marker gene(s) to allow for the selection of bacterial strains that have been transformed. Suitable selectable gene(s) or parts of selectable marker genes, include genes that render the bacteria resistant to drugs such as ampicillin, chloramphenicol, erythromycin, kanamycin, neomycin and tetracycline. Selectable markers also include biosynthetic genes, such as those in the histidine, tryptophan and leucine biosynthetic pathways.

Yeast selectable markers include the biosynthetic genes ADE2, HIS4, LEU2, and TRP1 when used in the context of auxotrophe strains; ALG7, which confers resistance to tunicamycin; the neomycin phosphotransferase gene, which confers resistance to G418; and the CUP1 gene, which allows yeast to grow in the presence of copper ions.

Suitable mammalian selection markers include, but are not limited to, those that confer resistance to neomycin (or its analog G418), blasticidin S, histinidol D, bleomycin, puromycin, hygromycin B, and other drugs. Selectable markers conferring survivability in a specific media include, but are not limited to Blasticidin S Deaminase, Neomycin phophotranserase 11, Hygromycin B phosphotranserase, Puromycin N-acetyl transferase, Bleomycin resistance protein (or Zeocin resistance protein, Phleomycin resistance protein, or phleomycin/zeocin binding protein), hypoxanthine guanosine phosphoribosyl transferase (HPRT), Thymidylate synthase, xanthine-guanine phosphoridosyl transferase, and the like.

Inclusion of Additional Elements

In a preferred embodiment, the expression vector contains an RNA splicing sequence upstream or downstream of the gene to be expressed in order to increase the level of gene expression. See Barret et al., Nucleic Acids Res. 1991; Groos et al., Mol. Cell. Biol. 1987; and Budiman et al., Mol. Cell. Biol. 1988.

In addition, the expression vector may comprise additional elements. For example, the expression vector may have two replication systems, thus allowing it to be maintained in two organisms, for example in mammalian or insect cells for expression and in a prokaryotic host for cloning and amplification. Furthermore, for integrating expression vectors, the expression vector contains at least one sequence homologous to the host cell genome, and preferably two homologous sequences which flank the expression construct. The integrating vector may be directed to a specific locus in the host cell by selecting the appropriate homologous sequence for inclusion in the vector. Such vectors may include cre-lox recombination sites, or attR, attB, attP, and attL sites. Constructs for integrating vectors and appropriate selection and screening protocols are well known in the art and are described in e.g., Mansour et al., Cell, 51:503 (1988) and Murray, Gene Transfer and Expression Protocols, Methods in Molecular Biology, Vol. 7 (Clifton: Humana Press, 1991).

In a preferred embodiment, the vector encodes a fusion protein, as discussed below.

Overview of Fusion Constructs

The library protein may also be made as a fusion protein, using techniques well known in the art. For example, fusion partners such as targeting sequences can be used which allow the localization of the library members into a subcellular or extracellular compartment of the cell. Purification tags may be fused with a library, allowing the purification or isolation of the library protein. Rescue sequences can be used to enable the recovery of the nucleic acids encoding them. Other fusion sequences are possible, such as fusions that enable utilization of a screening or selection technology.

Targeting or Signal Sequences

The expression vector may also include a signal peptide sequence that directs library protein and any associated fusions to a desired cellular location or to the extracellular media. For example some targeting sequences enable secretion of library protein in bacteria. The signal sequence typically encodes a signal peptide comprised of hydrophobic amino acids which direct the secretion of the protein from the cell, as is well known in the art. This method may be useful for gram-positive bacteria or gram-negative bacteria. The protein can be either secreted into the growth media or into the periplasmic space, located between the inner and outer membrane of the cell.

Suitable targeting sequences include, but are not limited to, binding sequences capable of causing binding of the expression product to a predetermined molecule or class of molecules while retaining bioactivity of the expression product, (for example by using enzyme inhibitor or substrate sequences to target a class of relevant enzymes); sequences signaling selective degradation, of itself or co-bound proteins; and signal sequences capable of constitutively localizing the candidate expression products to a predetermined cellular locale, including a) subcellular locations such as the Golgi, endoplasmic reticulum, nucleus, nucleoli, nuclear membrane, mitochondria, chloroplast, secretory vesicles, lysosome, and cellular membrane; and b) extracellular locations via a secretory signal. Target sequences also may be used in conjunction with cell surface display technology as discussed below.

Particularly preferred is localization to either subcellular locations or to the outside of the cell via secretion.

Purification Tags

In a preferred embodiment, the library member comprises a purification tag operably linked to the rest of the library peptide or protein. A purification tag is a sequence which may be used to purify or isolate the candidate agent, for detection, for immunoprecipitation, for FACS (fluorescence-activated cell sorting), or for other reasons. Thus, for example, purification tags include purification sequences such as polyhistidine, including but not limited to His6, or other tag for use with Immobilized Metal Affinity Chromatography (IMAC) systems (e.g. Ni+2 affinity columns), GST fusions, MBP fusions, Strep-tag, the BSP biotinylation target sequence of the bacterial enzyme BirA, and epitope tags which are targeted by antibodies. Suitable epitope tags include but are not limited to c-myc (for use with the commercially available 9E10 antibody), flag tag, and the like.

Rescue Fusions

A rescue fusion is a fusion protein that enables recovery of the nucleic acid encoding the library protein. In a preferred embodiment, such a rescue fusion would enable screening or selection of library members. Such fusion proteins may include but are not limited to, rep proteins, viral VPg proteins, transcription factors including but not limited to zinc fingers, RNA and DNA binding proteins, and the like. Attachment may be covalent or noncovalent.

Alternatively, the rescue sequence may be a unique oligonucleotide sequence that serves as a probe target site to allow the quick and easy isolation of the retroviral construct, via PCR, related techniques, or hybridization.

In an alternate embodiment, rescue sequences could also be based upon in vivo recombination systems, such as the cre-lox system, the Invitrogen Gateway system, forced recombination systems in yeast, mammalian, plant, bacteria or fungal cells (see WO 02/10183 A1), or phage display systems.

In an alternate embodiment, display technologies are utilized. For example, in phage display (see Kay, BK et al, eds. Phage display of peptides and proteins: a laboratory manual (Academic Press, San Diego, Calif., 1996); Lowman H B, Bass S H, Simpson N, Wells J A (1991) Selecting high-affinity binding proteins by monovalent phage display. Bioechemistry 30:10832-10838; Smith G P (1985) Filamentous fusion phage: novel expression vectors that display cloned antigens on the virion surface. Science 228:1315-1317.) library proteins can be fused to the gene III protein. Cell surface display (Witrrup K D, Protein engineering by cell-surface display. Curr. Opin. Biotechnology 2001, 12:395-399.) may also be useful for screening. This includes but is not limited to display on bacteria (see Georgiou G, Poetschke H L, Stathopoulos C, Francisco J A, Practical applications of engineering gram-negative bacterial cell surfaces. Trends Biotechnol. 1993 January;11 (1):6-10; Georgiou G, Stathopoulos C, Daugherty P S, Nayak A R, Iverson B L, and Curtiss R R (1997) Display of heterologous proteins on the surface of microorganisms: from the screening of combinatorial libraries to live recombinant vaccines. Nature Biotechnol. 15, 29-34; Lee J S, Shin K S, Pan J G, Kim C J. Surface-displayed viral antigens on Salmonella carrier vaccine. Nature Biotechnology, 2000, 18:645-648; June et al, 1998), yeast (see Boder E T, Wittrup K D: Yeast surface display for screening combinatorial polypeptide libraries. Nat Biotechnol 1997,15:553-557 Boder ET and Wittrup K D. Yeast surface display for directed evolution of protein expression, affinity, and stability. Methods Enzymol 2000, 328:430-44.), and mammalian cells (see Whitehorn E A, Tate E, Yanofsky S D, Kochersperger L, Davis A, Mortensen R B, Yonkovich S, Bell K, Dower W J, and Barrett R W 1995. A generic method for expression and use of “tagged” soluble versions of cell surface receptors. Bio/technology, 13,1215-1219.).

Additional Fusions that Allow for Screening or Selection

In an alternate embodiment, a protein fragment complementation assay is used (see Johnsson N & Varshavsky A. Split Ubiquitin as a sensor of protein interactions in vivo. 1994 Proc Natl Acad Sci USA, 91: 10340-10344; Pelletier J N, Campbell-Valois F X, Michnick S W. Oligomerization domain-directed reassembly of active dihydrofolate reductase from rationally designed fragments. 1998. Proc Natl Acad Sci USA 95:12141-12146.) Other fusion methods which may allow screening include but are not limited to periplasmic expression and cytometric screening (see Chen G, Hayhurst A, Thomas J G, Harvey B R, Iverson B L, Georgiou G: Isolation of high-affinity ligand-binding proteins by periplasmic expression with cytometric screening (PECS). Nat Biotechnol 2001, 19: 537-542.), and the yeast two hybrid screen (see Fields S, Song O: A novel genetic system to detect protein-protein interactions. Nature 1989, 340:245-246.)

Additional fusion partners may also be utilized. Thus, for example, for the creation of monoclonal antibodies, if the desired epitope is small, the library protein may be fused to a carrier protein to form an immunogen. Alternatively, the library protein may be made as a fusion protein to increase expression, or for other reasons. For example, when the library protein is a library peptide, the nucleic acid encoding the peptide may be linked to other nucleic acid for expression purposes. Similarly, other fusion partners may be used, such as targeting sequences which allow the localization of the library members into a subcellular or extracellular compartment of the cell, rescue sequences or purification tags which allow the purification or isolation of either the library protein or the nucleic acids encoding them; stability sequences, which confer stability or protection from degradation to the library protein or the nucleic acid encoding it, for example resistance to proteolytic degradation, or combinations of these, as well as linker sequences as needed.

In a preferred embodiment, the fusion partner is a stability sequence to confer stability to the library member or the nucleic acid encoding it. Thus, for example, peptides may be stabilized by the incorporation of glycines after the initiation methionine (MG or MGG), for protection of the peptide to ubiquitination as per Varshavsky's N-End Rule, thus conferring long half-life in the cytoplasm. Similarly, two prolines at the C-terminus impart peptides that are largely resistant to carboxypeptidase action. The presence of two glycines prior to the prolines impart both flexibility and prevent structure initiating events in the di-proline to be propagated into the candidate peptide structure. Thus, preferred stability sequences are as follows: MG(X)nGGPP, where X is any amino acid and n is an integer of at least four.

Linkers

Linker sequences may be used to connect the library protein to its fusion partner or tag. The linker sequence will generally comprise a small number of amino acids, typically less than ten. However, longer linkers may also be used. As will be appreciated by those skilled in the art, any of a wide variety of sequences may be used as linkers. Typically, linker sequences are selected to be flexible and resistant to degradation. A common linker sequence comprises the amino acid sequence GGGGS. The preferred linker between a protein and C-terminal PP tag consists of two glycines.

Labels

In one embodiment, the library nucleic acids, proteins and antibodies of the invention are labeled. In general, labels fall into three classes: a) immune labels, which may be an epitope incorporated as a fusion constructs may which is recognized by an antibody as discussed above, isotopic labels, which may be radioactive or heavy isotopes, and c) small molecule labels which may include fluorescent and colorimetric dyes or molecules such as biotin which enable the use of other labeling techniques. Labels may be incorporated into the compound at any position and may be incorporated in vivo during protein or peptide expression or in vitro.

Transformation and Transfection Methods

The methods of introducing exogenous nucleic acid into host cells is well known in the art, and will vary with the host cell used. Techniques include dextran-mediated transfection, calcium phosphate precipitation, calcium chloride treatment, polybrene mediated transfection, protoplast fusion, electroporation, viral or phage infection, encapsulation of the polynucleotide(s) in liposomes, and direct microinjection of the DNA into nuclei. In the case of mammalian cells, transfection may be either transient or stable.

Expression Systems

The library proteins of the present invention are produced by culturing a host cell transformed with nucleic acid, preferably an expression vector, containing nucleic acid encoding an library protein, under the appropriate conditions to induce or cause expression of the library protein. As outlined below, the libraries may be the basis of a variety of display techniques, including, but not limited to, phage and other viral display technologies, yeast, bacterial, and mammalian display technologies. The conditions appropriate for library protein expression will vary with the choice of the expression vector and the host cell, and will be easily ascertained by one skilled in the art through routine experimentation. For example, the use of constitutive promoters in the expression vector will require optimizing the growth and proliferation of the host cell, while the use of an inducible promoter requires the appropriate growth conditions for induction. In addition, in some embodiments, the timing of the harvest is important. For example, the baculoviral systems used in insect cell expression are lytic viruses, and thus harvest time selection may be crucial for product yield.

As will be appreciated by those in the art, the type of cells used in the present invention may vary widely. Basically, a wide variety of appropriate host cells may be used, including yeast, bacteria, archaebacteria, fungi, and insect and animal cells, including mammalian cells. Of particular interest are Drosophila melanogaster cells, Saccharomyces cerevisiae and other yeasts, E. coli, Bacillus subtilis, SF9 cells, C129 cells, 293 cells, Neurospora, BHK, CHO, COS, and HeLa cells, fibroblasts, Schwanoma cell lines, immortalized mammalian myeloid and lymphoid cell lines, Jurkat cells, mast cells and other endocrine and exocrine cells, and neuronal cells. See the ATCC cell line catalog, hereby expressly incorporated by reference. In addition, the expression of the secondary libraries in phage display systems, such as are well known in the art, are particularly preferred, especially when the secondary library comprises random peptides. In one embodiment, the cells may be genetically engineered, that is, contain exogenous nucleic acid, for example, to contain target molecules.

Mammalian Expression Systems

In a preferred embodiment, the library proteins are expressed in mammalian cells. Any mammalian cells may be used, with mouse, rat, primate and human cells being particularly preferred, although as will be appreciated by those in the art, modifications of the system by pseudotyping allows all eukaryotic cells to be used, preferably higher eukaryotes. As is more fully described below, a screen will be set up such that the cells exhibit a selectable phenotype in the presence of a random library member. As is more fully described below, cell types implicated in a wide variety of disease conditions are particularly useful, so long as a suitable screen may be designed to allow the selection of cells that exhibit an altered phenotype as a consequence of the presence of a library member within the cell.

Accordingly, suitable mammalian cell types include, but are not limited to, tumor cells of all types (particularly melanoma, myeloid leukemia, carcinomas of the lung, breast, ovaries, colon, kidney, prostate, pancreas and testes), cardiomyocytes, endothelial cells, epithelial cells, lymphocytes (T-cell and B cell) , mast cells, eosinophils, vascular intimal cells, hepatocytes, leukocytes including mononuclear leukocytes, stem cells such as haemopoietic, neural, skin, lung, kidney, liver and myocyte stem cells (for use in screening for differentiation and de-differentiation factors), osteoclasts, chondrocytes and other connective tissue cells, keratinocytes, melanocytes, liver cells, kidney cells, and adipocytes. Suitable cells also include known research cells, including, but not limited to, Jurkat T cells, NIH3T3 cells, CHO, COS, etc. See the ATCC cell line catalog, hereby expressly incorporated by reference.

Mammalian expression systems are also known in the art, and include retroviral systems. A mammalian promoter is any DNA sequence capable of binding mammalian RNA polymerase and initiating the downstream (3′) transcription of a coding sequence for library protein into mRNA. A promoter will have a transcription-initiating region, which is usually placed proximal to the 5′ end of the coding sequence, and a TATA box, usually located 25-30 base pairs upstream of the transcription initiation site. The TATA box is thought to direct RNA polymerase 11 to begin RNA synthesis at the correct site. A mammalian promoter will also contain an upstream promoter element (enhancer element), typically located within 100 to 200 base pairs upstream of the TATA box. An upstream promoter element determines the rate at which transcription is initiated and may act in either orientation. Of particular use as mammalian promoters are the promoters from mammalian viral genes, since the viral genes are often highly expressed and have a broad host range. Examples include the SV40 early promoter, mouse mammary tumor virus LTR promoter, adenovirus major late promoter, herpes simplex virus promoter, and the CMV promoter.

Typically, transcription termination and polyadenylation sequences recognized by mammalian cells are regulatory regions located 3′ to the translation stop codon and thus, together with the promoter elements, flank the coding sequence. The 3′ terminus of the mature mRNA is formed by site-specific post-translational cleavage and polyadenylation. Examples of transcription terminator and polyadenylation signals include those derived from SV40, and the like.

The methods of introducing exogenous nucleic acid into mammalian hosts, as well as other hosts, is well known in the art, and will vary with the host cell used. Techniques include dextran-mediated transfection, calcium phosphate precipitation, polybrene mediated transfection, protoplast fusion, electroporation, viral infection, encapsulation of the polynucleotide(s) in liposomes, and direct microinjection of the DNA into nuclei.

Bacterial Expression Systems

In a preferred embodiment, library proteins are expressed in bacterial systems. Bacterial expression systems are well known in the art and include Bacillus subtilis, E. coli, Streptococcus cremoris, and Streptococcus lividans.

A suitable bacterial promoter is any nucleic acid sequence capable of binding bacterial RNA polymerase and initiating the downstream (3′) transcription of the coding sequence of library protein into mRNA. A bacterial promoter has a transcription initiation region which is usually placed proximal to the 5′ end of the coding sequence. This transcription initiation region typically includes an RNA polymerase binding site and a transcription initiation site. Sequences encoding metabolic pathway enzymes provide particularly useful promoter sequences. Examples include promoter sequences derived from sugar metabolizing enzymes, such as galactose, lactose and maltose, and sequences derived from biosynthetic enzymes such as tryptophan. Promoters from bacteriophage may also be used and are known in the art. In addition, synthetic promoters and hybrid promoters are also useful; for example, the tac promoter is a hybrid of the trp and lac promoter sequences. Furthermore, a bacterial promoter may include naturally occurring promoters of non-bacterial origin that have the ability to bind bacterial RNA polymerase and initiate transcription.

In addition to a functioning promoter sequence, an efficient ribosome-binding site is desirable. In E. coli, the ribosome-binding site is called the Shine-Dalgarno (SD) sequence and includes an initiation codon and a sequence 3-9 nucleotides in length located 3-11 nucleotides upstream of the initiation codon.

Baculovirus Expression System

In one embodiment, library proteins are produced in insect cells, including but not limited to Drosophila melanogaster S2 cells, as well as cells derived from members of the order Lepidoptera which includes all butterflies and moths, such as the silkmoth Bombyx mori and the alphalpha looper Autographa californica. Lepidopteran insects are host organisms for some members of a family of virus, known as baculoviruses (more than 400 known species), that infect a variety of arthropods. (see U.S. Pat. No. 6,090,584).

Expression vectors for the transformation of insect cells, and in particular, baculovirus-based expression vectors, are well known in the art and are described e.g., in O'Reilly et al., Baculovirus Expression Vectors: A Laboratory Manual (New York: Oxford University Press, 1994).

In an alternate embodiment, library proteins are produced in insect cells. The library may be transfected into SF9 Spodoptera frugiperda insect cells to generate baculovirus which are used to infect SF21 or High Five commercially available from Invitrogen, insect cells for high level protein production. Also, transfections into the Drosophila Schneider S2 cells will express proteins.

Yeast Expression Systems

In a preferred embodiment, library protein is produced in yeast cells. Yeast expression systems are well known in the art, and include expression vectors for Saccharomyces cerevisiae, Candida albicans and C. maltosa, Hansenula polymorpha, Kluyveromyces fragilis and K. lactis, Pichia guillerimondii and P. pastoris, Schizosaccharomyces pombe, and Yarrowia lipolytica. Preferred promoter sequences for expression in yeast include the inducible GAL1,10 promoter, the promoters from alcohol dehydrogenase, enolase, glucokinase, glucose-6-phosphate isomerase, glyceraldehyde-3-phosphate-dehydrogenase, hexokinase, phosphofructokinase, 3-phosphoglycerate mutase, pyruvate kinase, and the acid phosphatase gene. Yeast selectable markers include, but are not limited to ADE2, HIS4, LEU2, TRP1, and ALG7, which confers resistance to tunicamycin; the neomycin phosphotransferase gene, which confers resistance to G418; and the CUP1 gene, which allows yeast to grow in the presence of copper ions.

In Vitro Expression Systems

In one embodiment, the library proteins are expressed in vitro using cell-free translation systems. Several commercial sources are available for this system including but not limited to Roche Rapid Translation System, Promega TnT system, Novagen's EcoPro system, Ambion's ProteinScipt-Pro system. In vitro translation systems derived from both prokaryotic (e.g. E. coli) and eukaryotic (e.g. Wheat germ, Rabbit reticulocytes) cells are available and may be chosen based on the expression levels and functional properties of the protein of interest. Both linear (as derived from a PCR amplification) and circular (as in plasmid) DNA molecules are suitable for such expression as long as they contain the gene encoding the protein operably linked to an appropriate promoter. Other features of the molecule that are important for optimal expression in either the bacterial or eukaryotic cells (including the ribosome binding site etc) are also included in these constructs. The proteins may again be expressed individually or in suitable size pools consisting of multiple library members. The main advantage offered by these in vitro systems is their speed and ability to produce soluble proteins. In addition the protein being synthesized may be selectively labeled if needed for subsequent functional analysis.

Protein Purification

In a preferred embodiment, the library protein is purified or isolated after expression. Library proteins may be isolated or purified in a variety of ways known to those skilled in the art depending on what other components are present in the sample. The degree of purification necessary will vary depending on the use of the library protein. In some instances no purification will be necessary. For example in one embodiment, if library proteins are secreted, screening or selection may take place directly from the media.

Standard purification methods include electrophoretic, molecular, immunological and chromatographic techniques, including ion exchange, hydrophobic, affinity, size exclusion chromatography, and reversed-phase HPLC chromatography, as well as precipitation, dialysis, and chromatofocusing techniques. Purification can often be facilitated by the inclusion of purification tag, as described above. For example, the library protein may be purified using glutathione resin if a GST fusion is employed, Immobilized Metal Affinity Chromatography (IMAC) if a His or other tag is employed, or immobilized anti-flag antibody if a flag tag is used. Ultrafiltration and diafiltration techniques, in conjunction with protein concentration, are also useful. For general guidance in suitable purification techniques, (see Scopes, R., Protein Purification: Principles and Practice 3rd Ed., Springer-Verlag, N.Y. (1994).), hereby expressly incorporated by reference.

In a preferred embodiment, the libraries are used in any number of display techniques. For example, the libraries may be displayed using phage or enveloped virus systems, bacterial systems, yeast two hybrid systems or mammalian systems.

In a preferred embodiment, the libraries are displayed using a phage or enveloped virus system. For example, a library of viruses, each carrying a distinct peptide sequence as part of the coat protein, can be produced by inserting random oligonucleotides sequences into the coding sequence of viral coat or envelope proteins. Several different viral systems have been used to display peptides, as described in Smith, G.P, (1985) Science, 228:1315-1317; Santini, C., et al., (1998) J. Mol. Biol., 282:125-135; Sternberg, N. and Hoess, R. H. (1995) Proc. Natl. Acad. Sci. USA, 92:1609-1613; Maruyama, I. N., et al. (1994) Proc. Natl. Acad. Sci. USA, 91:8273-8277; Dunn, I. S., (1995) J. Mol. Biol., 248:497-506; Rosenberg, A., et al. (1996) Innovations 6:1-6); Ren, Z. J., et al. (1996) Protein Sci., 5:1833-1843; Efimov, V. P., et al. (1995) Virus Genes 10:173-177; Dulbecco, R., U.S. Pat. No. 4,593,002; Ladner, R. C.., et al., U.S. Pat. No. 5,837,500; Ladner, R. C., et al., U.S. Pat. No. 5,223,409; Dower, et al., U.S. Pat. No. 5,427,908; Russell et al., U.S. Pat. No. 5,723,287; Li U.S. Pat. No. 6,190,856; U.S. Ser. No. 10/218,102, and the application entitled “METHODS AND COMPOSITIONS FOR THE CONSTRUCTION AND USE OF ENVELOPE VIRUSES AS DISPLAY PARTICLES”, filed Aug. 2, 2001, serial number not yet assigned, all of which are expressly incorporated by reference.

In a preferred embodiment, the libraries are displayed on the surface of a bacterial cell as is described in WO 97/37025, which is expressly incorporated by reference in its entirety. In this embodiment, surface anchoring vectors are provided for the surface expression of genes encoding proteins of interest. At a minima, the vector includes a gene encoding an ice nucleation protein, a secretion signal a targeting signal and a gene of interest. Preferably, the bacterial host is a gram negative bacterium belonging to the genera Escherichia, Acetobacter, Pseudomonas, Xanthomonas, Erwinia, and Xymomonas. Advantages to using the ice nucleation protein as the surface anchoring protein are the high level of expression of the ice nucleation protein on the surface of the bacterial cell and its stable expression during the stationary phase of bacterial cell growth.

In a preferred embodiment, the libraries are displayed using yeast-based, two-hybrid systems as is described in Fields and Song (1989) Nature 340:245, which is expressly incorporated herein by reference. Yeast-based, two-hybrid systems utilize chimeric genes and detect protein-protein interactions via the activation of reporter-gene expression. Reporter-gene expression occurs as a result of reconstitution of a functional transcription factor caused by the association of fusion proteins encoded by the chimeric genes. Preferably, the yeast-based, two-hybrid system commercially available from Clontech is used to screen libraries for proteins that interact with a candidate proteins. See generally, Ausubel et al., Current Protocols in Molecular Biology, John Wiley & Sons, pp.13.14.1-13.14.14, which is expressly incorporated herein by reference.

In a preferred embodiment, the libraries are displayed using mammalian systems. For example, a cell-based display can be used to display large cDNA libraries in mammalian cells as described in Nolan, et al., U.S. Pat. No. 6,153,380; Shioda , et al. U.S. Pat. No. 6,251,676, both of which are expressly incorporated herein by reference.

Screening of Libraries

Library members may be screened using a variety of assays, including but not limited to in vitro assays and in vivo assays such as cell-based, tissue-based, and whole-organism assays. Automation and high-throughput screening technologies may be utilized in the screening procedures.

High-Throughput Screening Technology

Fully robotic or microfluidic systems include automated liquid-, particle-, cell- and organism-handling including high throughput pipetting to perform all steps of experimental library generation, protein expression, and library screening. This includes liquid, particle, cell, and organism manipulations such as aspiration, dispensing, mixing, diluting, washing, accurate volumetric transfers; retrieving, and discarding of pipette tips; and repetitive pipetting of identical volumes for multiple deliveries from a single sample aspiration. These manipulations are cross-contamination-free liquid, particle, cell, and organism transfers. This instrument performs automated replication of microplate samples to filters, membranes, and/or daughter plates, high-density transfers, full-plate serial dilutions, and high capacity operation.

In addition, as will also be appreciated by those in the art, biochips may be part of the HTS system utilizing any number of components such as biosensor chips with protein arrays to measure protein-protein interactions or DNA-sensor chips to measure protein-DNA interactions. Microfluidic chip arrays (e.g., those commercially available from Caliper) may also be utilized in the context of automated HTS screening.

The automated HTS system used may include a computer workstation comprising a microprocessor programmed to manipulate a device selected from the group consisting of a thermocycler, a multichannel pipetter, a sample handler, a plate handler, a gel loading system, an automated transformation system, a gene sequencer, a colony picker, a bead picker, a cell sorter, an incubator, a light microscope, a fluorescence microscope, a spectrofluorimeter, a spectrophotometer, a luminometer, a CCD camera and combinations thereof.

In Vivo Screening

In a preferred embodiment, the library is screened using in vivo assay systems, including cell-based, tissue-based, or whole-organism assay systems. Cells, tissues, or organisms may be exposed to individual library members or pools containing several library members. Alternatively, host cells may be transformed or transfected with DNA encoding the library proteins and analyzed for phenotypic alterations.

A variety of other reagents may be included in the assays. These include reagents like salts, neutral proteins, e.g. albumin, detergents, etc which may be used to facilitate optimal protein-protein binding and/or reduce non-specific or background interactions. Also reagents that otherwise improve the efficiency of the assay, such as protease inhibitors, nuclease inhibitors, anti-microbial agents, etc., may be used. The mixture of components may be added in any order that provides for detection. Washing or rinsing the cells will be done as will be appreciated by those in the art at different times, and may include the use of filtration and centrifugation. When second labeling moieties (also referred to herein as “secondary labels”) are used, they are preferably added after excess non-bound target molecules are removed, in order to reduce non-specific binding; however, under some circumstances, all the components may be added simultaneously.

To screen the library, experimental systems are developed in which the activity for the library protein of interest is coupled to an observable property. Typical observable properties include changes in absorbance, fluorescence, or luminescence. Screens may also monitor changes in properties such as cell morphology or viability, and the like.

For example, cell death or viability may be measured using dyes or immuno-cytochemical reagents (e.g. Caspase staining assay for apoptosis, Alamar blue for cell vitality) that specifically recognize either viable or inviable cells.

In an alternate cell death or viability assay, the cells are transformed or transfected with a receptor or binding partner protein responsive to the ligand represented by the library. The receptor may be coupled to a signaling pathway that causes cell death, allows cell survival, or triggers expression of a reporter gene. These readout modalities can be measured using dyes or immuno-cytochemical reagents that indicate cell death, cell vitality (e.g. Caspase staining assay for apoptosis, Alamar blue for cell vitality).

Alternatively, readout may be via a reporter construct. Reporter constructs may be proteins that are intrinsically fluorescent or colored, or proteins that modify the spectral properties of a substrate or binding partner. Common reporter constructs include the proteins luciferase, green fluorescent protein, and beta-galactosidase.

The assays described may also be performed by measuring morphological changes of the cells as a response to the presence of a library variant. These morphological changes may be registered using microscopic image analysis systems (e.g. Cellomics ArrayScan technology) such as those now available commercially.

In Vitro Screening

In a preferred embodiment, different physical and functional properties of the library members are screened in an in vitro assay. Properties of library members that may be screened include, but are not limited to, various aspects of stability (including pH, thermal, oxidative/reductive and solvent stability), solubility, affinity, activity and specificity. Multiple properties can be screened simultaneously (e.g. substrate specificity in organic solvents, receptor-ligand binding at low pH) or individually.

Protein properties may be assayed and detected in a wide variety of ways. Typical readouts include, but are not limited to, chromogenic, fluorescent, luminescent, or isotopic signals. These detection modalities are utilized in several assay methods including, but not limited to, FRET (fluorescence resonance energy transfer) and BRET (bioluminescence resonance energy transfer) based assays, AlphaScreen (Amplified Luminescent Proximity Homogeneous Assay), SPA (scintillation proximity assay), ELISA (enzyme-linked immunosorbent assays), BIACORE (surface plasmon resonance), or enzymatic assays. In vitro screening may or may not utilize a protein fusion or a label.

Selection of Libraries

In an alternatively preferred embodiment, a selection method is used to select for desired library members. This is generally done on the basis of desired phenotypic properties, e.g. the protein properties defined herein. This is enabled by any method which couples phenotype and genotype, i.e. protein function with the nucleic acid that codes for it. In some cases this will be a “trans” effect rather than a “cis” effect. In this way, isolation of library protein variants simultaneously enables isolation of its coding nucleic acid. Once isolated, the gene or genes encoding library protein can be purified (“rescued”) and/or amplified. This process of isolation and amplification can be repeated, allowing favorable protein variants in the library to be enriched. Nucleic acid sequencing of the selected library members ultimately allows for identification of library members with desired properties.

Isolation of library protein may be accomplished by a number of methods. In some embodiments, only cells containing library protein variants with desired protein properties are allowed to survive or replicate. In alternate embodiments, the library protein and its genetic material are obtained by binding the library protein to another protein, RNA aptamer, or other molecule.

In one embodiment, the selection method is based on the use of specific fusion constructs. For example, if phage display is used, the library members are fused to the phage gene III protein.

In one embodiment selection is accomplished using a rescue fusion sequence, which forms a covalent or noncovalent link between the library member (phenotype) and the nucleic acid that encodes the library member (genotype). For example, in a preferred embodiment the rescue fusion protein binds to a specific sequence on the expression vector (see U.S. Ser. No. 09/642,574; PCT/US00/22906; U.S. Ser. No. 10/023,208; PCT/US01/49058; U.S. Ser. No. 09/792,630; U.S. Ser. No. 10/080,376; PCT/US02/04852; U.S. Ser. No. 09/792,626; PCT/US02/04853; U.S. Ser. No. 10/082,671; U.S. Ser. No. 09/953,351; PCTUS01/28702; U.S. Ser. No. 10/097,100; and PCT/US02/07466), and envelope virus (see U.S. Ser. No. 09/922,503 and PCT/US01/24535).

In an alternate embodiment, in vitro selection methods that do not rely on display technologies are used. These methods include, but are not limited to, periplasmic expression and cytometric screening (see Chen G, Hayhurst A, Thomas J G, Harvey B R, Iverson B L, Georgiou G: Isolation of high-affinity ligand-binding proteins by periplasmic expression with cytometric screening (PECS). Nat Biotechnol 2001,19: 537-542), protein fragment complementation assay (see Johnsson N & Varshavsky A. Split Ubiquitin as a sensor of protein interactions in vivo. (1994) Proc Natl Acad Sci USA, 91:10340-10344.) and the yeast-based, two-hybrid screen (see Fields S, Song O: A novel genetic system to detect protein-protein interactions. Nature 1989, 340:245-246.) used in selection mode (see Visintin M, Tse E, Axelson H, Rabbitts T H, Cattaneo A: Selection of antibodies for intracellular function using a two-hybrid in vivo system. Proc Natl Acad Sci USA 1999, 96: 11723-11728.).

In an alternative embodiment, in vivo selection may occur if expression of the library protein imparts some growth, reproduction, or survival advantage to the cell. For example, if host cells transformed with a library comprising variants of an essential enzyme are grown in the presence of the corresponding substrate; only clones with a functional variant of the enzyme will survive. Alternatively, an advantage may be conferred if the library member comprises a growth or survival factor and the host cell expresses the appropriate receptor.

Additional Characterization

In a preferred embodiment, a library member or members isolated using some screening or selection method are further characterized. The library member(s) may be subjected to further biological, physical, structural, kinetic, and thermodynamic analysis. Thus, for example, a selected library variant may be subjected to physical-chemical characterization using gel electrophoresis, reversed-phase HPLC, SEC-HPLC, mass spectrometry (MS) including but not limited to LC-MS, LC-MS peptide mapping and the like, ultraviolet absorbance spectroscopy, fluorescence spectroscopy, circular dichroism spectroscopy, isothermal titration calorimetry, differential scanning calorimetry, surface plasmon resonance, analytical ultra-centrifugation, proteolysis, and cross-linking. Structural analysis employing X-ray crystallographic techniques and nuclear magnetic resonance spectroscopy are also useful. As is known to those skilled in the art, several of the above methods may also be used to determine the kinetics and thermodynamics of binding and enzymatic reactions. The biological properties of one or more library members, including pharmacokinetics and toxicity, may also be characterized in cell, tissue, and whole organism experiments.

Modification of Libraries to Generate Further Libraries

In a preferred embodiment, a variety of additional steps may be done to generate additional libraries, i.e., secondary, tertiary, etc., from the protein libraries created using the RNA shuffling techniques described above. For example, computational processing and/or additional recombination approaches may be used to generate additional libraries.

Computational Approaches to Library Generation

In essence, any computational method may be used to generate additional libraries. For example, sequence and/or structural alignment programs, energy calculation methods (i.e., force-field calculations), electrostatic models, scoring functions, a protein design algorithm, a sequence prediction algorithm, other inverse folding methods, molecular dynamics calculations, as well as other computational methods such as combinatorial optimization, Taboo algorithms and Clustering algorithms may be-used (see U.S. Ser. No. 10/218,102, incorporated herein by reference in its entirety).

In a preferred embodiment, a protein design algorithm (PDA™) is used to generate additional protein sequences as is described in U.S. Pat. Nos. 6,269,312, 6,188,965, and 6,403,312, and are herein expressly incorporated by reference.

In a preferred embodiment, a sequence prediction algorithm (SPA) is used to generate additional protein sequences as is described in Raha, K., et al. (2000) Protein Sci., 9: 1106-1119, U.S. Ser. No. 09/877,695; U.S. Ser. No. to be determined for a continuation-in-part application filed on Feb. 6, 2002, entitled APPARATUS AND METHOD FOR DESIGNING PROTEINS AND PROTEIN LIBRARIES, with John R. Desjarlais as inventor, expressly incorporated herein by reference.

Additional Experimental Approaches to Library Generation

Additional variability can be added to the tertiary library as well, either experimentally (e.g. through the use of error-prone PCR in tertiary library sequences) or computationally (adding an “in silico” variant generation step to sample more sequence space). In the latter case, it is possible to introduce this additional level of variability in a random fashion (as used herein random includes variation introduced in a controlled manner or an uncontrolled manner) or in a directed fashion. For example, directed variability may be introduced by adding certain residues from a particular sequence, e.g. the human sequence. See for example U.S. Pat. No. 6,403,312, U.S. Ser. Nos. 09/782,004, 09/927,790, 10/101,499, 10/218,102, Stemmer, et al. (1994) Nature 370:389-391; Stemmer, et al., (1994) Proc. Natl. Acad. Sci. USA, 91:10747-10751; U.S. Pat. Nos. 5,603,793, 5,830721, 5,811,238, and U.S. Pat. No. 6,426,224; all of which are incorporated herein by reference in their entirety.

Once made find use in a number of applications.

The following examples serve to more fully describe the manner of using the above-described invention, as well as to set forth the best modes contemplated for carrying out various aspects of the invention. It is understood that these examples in no way serve to limit the true scope of this invention, but rather are presented for illustrative purposes. All references cited herein are incorporated by reference.

### EXAMPLES

### Example 1

### Recombination Between Beta Lactamase Variants

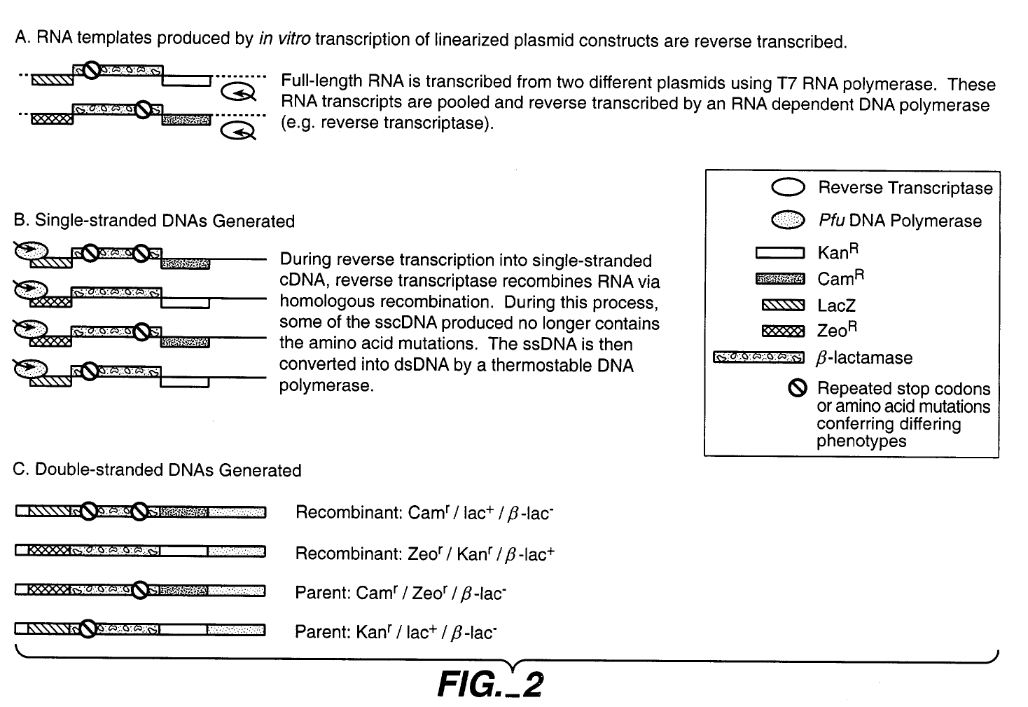

This example demonstrates how the RNA-dependent DAN polymerase MMLV reverse transcriptase can undergo template switching between homologous sequences, two genes encoding beta lactamase variants that each differ from the wild-type sequence by a single amino acid mutation. the polynucleotide constructs used contain gene markers flanking the beta lactamase gene to easily distinguish whether a template switch has occurred. Crossover from one template to the other results in the acquisition of the lacZ alpha gene and the loss of the zeocin resistance gene or vice versa depending on which template serves as the initiation template for reverse transcription (see FIG. 2).

Vector Construction

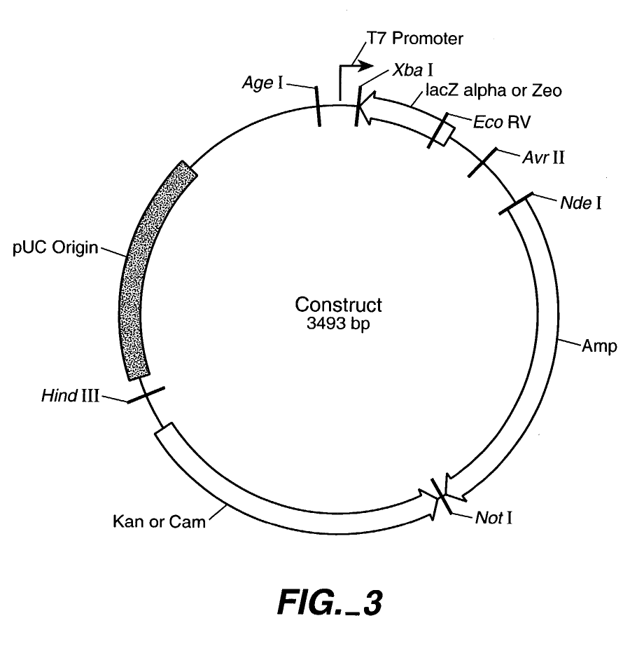

The plasmid vector pETBlue-1, commercially available from Novagen is used as the basis for generating vectors pKAL and pCAZ (see FIG. 3).

pKAL Vector

The BspHI site of pETBlue-1 is replaced with a HindIII site using the Quikchange site-directed mutagenesis kit, commercially available from Stratagene. Digestion with HindIII and AvrII is used to isolate a fragment containing the pUC origin, T7 promoter and the lacza. The b-lactamase gene (Amp) gene, modified with an NdeI restriction site at the start codon, plus flanking sequences to include the promoter was PCR amplified from vector pBAD/HisMycB, commercially available from Invitrogen. The PCR primers included a 5′ AvrII restriction site and a 3′ NotI site. The kanamycin resistance gene (Kan) with some flanking sequences was amplified from pET24a+ vector, commercially available from Novagen, with ends containing a 5′ HindIII and 3′ NotI restriction sites. The three fragments containing the origin of replication, the Amp gene, and the Kan gene were ligated together to form vector pKAL.

pCAZ Vector

The chloramphenical resistance gene (Cm) was PCR amplified from pLysS vector, commercially available from Novagen, with 5′ HindIII and 3′ NotI site restriction sites. The zeocin (Zeo) gene with the EM7 promoter from pEM7/Zeo vector, commercially available from Invitrogen, was isolated by digestion with EcoRV and XbaI restriction enzymes. Vector pKAL was digested with HindIII and XbaI restriction enzymes to isolate the origin of replication and the T7 promoter The three fragments containing the origin of replication, the Cm gene, and the Zeo gene were ligated together to form vector pCAZ

Genes for Recombining

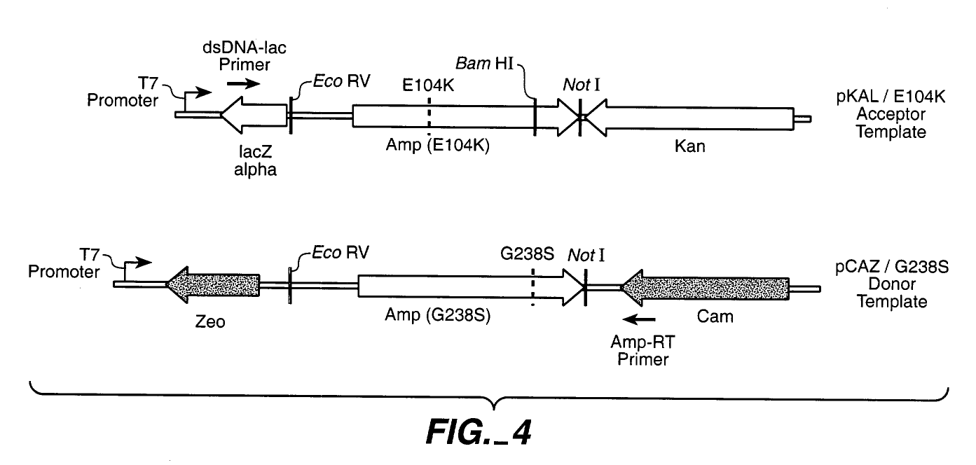

Two different b-lactamase variants were sub-cloned into the pKAL and pCAZ vector constructs (see FIG. 4). Construct pKAL/E104K was made by replacing the Amp gene from pKAL between NdeI and NotI with a b-lactamase gene variant which differs from the wild-type sequence at amino acid position 104. A glutamate residue replaces the lysine (E104K). Construct pCAZ/G238S was made by replacing the Amp gene from pCAZ between NdeI and NotI with a b-lactamase gene variant which differs from the wild-type sequence at amino acid position 238. A glycine residue replaces the serine (G238S). The E104K variant also contains a silent BamHI site not present in G238S.

Template Linearization/Purification

pKAL/E014K and pCAZ/G238S were digested at a unique Agel restriction site (see FIG. 3) to leave a 5′ overhang. The digest conditions in a 30 microliter reaction volume are: 10 μg of plasmid DNA, 6 U Agel, commercially available from New England Biolabs, 10 mM Bis Tris Propane-HCl (pH 7.0), 10 mM MgCl₂, 1 mM DTT incubated at 25° C. overnight. After digestion, the reaction was treated with proteinase K (100 μg/ml) and 0.5% SDS for 1 hr at 50° C. This was followed by phenol/chloroform extraction and ethanol precipitation.

In Vitro Transcription

In vitro transcriptions were done using the T7 MEGAscript High Yield transcription Kit, commercially available from Ambion. The reactions were set-up according to manufacturer's protocol using 1 μg of the linearized constructs prepared above in a 20 μl volume. Reactions were incubated for 2 hr at 30° C. followed by a 2 min incubation at 65° C. Two units (U) of DNAsel were added and the reaction was further incubated at 37° C. for 1 hour. The RNA was purified by acid-pheno/chloroform extraction and ethanol precipitation. RNA integrity was analyzed by denaturing agarose gel electrophoresis and quantified by spectrophotometry.

Reverse Transcription

Reverse transcription was done in 25 mM Tris-HCl (pH 8.3), 75 mM KCl, 3 mM MgCl₂, 10 mM DTT, 0.5 mM dNTPs, 40 Units of RNaseOUT inhibitor, commercially available from Invitrogen in a 20 μl volume. Annealing of 2 pmole of primer Amp-RT to 50 pmole total RNA template (25 pmol pKAL/E104K and 25 pmole pCAZ/G238S) was obtained by heating the reaction mixture without buffer, DTT and RNaseOUT to 94° C. for 1 minute followed by slow cooling to 37° C. DTT, buffer and RNaseOUT were added and the reaction started by the addition of 400 U MMLV reverse transcriptase, commercially available from Invitrogen. Incubation at 37° C. proceeds for 1.5 hours. The addition of 0.5 μg DNase-free Rnase, commercially available from Roche and 1 U Rnase H, commercially available from Roche is followed by incubation at 37° C. for 30 minutes.

PCR

PCR is done to amplify single stranded DNA produce above into double stranded DNA. The recombination specific PCR primers Amp-RT and dsDNA-lac were used (see FIG. 4 for annealing sites). Two μl of the reverse transcriptase reaction from above was added to the PCR mix (20 mM Tris-HCL (pH8.8), 2 mM MgSO₄, 10 mM KCl, 10 mM (NH₄)₂SO₄, 0.1% Triton X-100, 0.1 mg/ml nuclease-free BSA, 0.3 mM dNTPs). 1.25 U Platinum Pfx DNA polymerase, commercially available from Invitrogen was added. The final reaction volume is 50 μl. The PCR cycling conditions are 94° C. for 5 minutes; 30 cycles at 94° C. for 30 seconds; 55° C. for 1 minute; 68° C. for 2 minutes; and a final extension at 68° C. for 7 minutes.

Cloning and Analysis

PCR band products corresponding to the expected size were cloned into pCR-BluntII-Topo vector, commercially available from Invitrogen, and plated on kanamycin and X-gal containing LB agar plates. Colonies were randomly picked for sequencing on a MegaBACE 1000, commercially available from Amersham.

Sequencing Results

The sequencing results are shown in Table 1. When reverse transcription was initiated with a 10 sequence specific primer which anneals to the chloramphenical resistance gene of the pCAZ/G238S construct, pCAZ/G238S served as the donor template during reverse transcription. If no template switching occurs, it would be expected that the products would not have the lacZ alpha gene. However, if template switching occurs within the homologous region between EcoRV and NotI (see FIG. 4) of the two templates (pKAL/E104K and pCAZ/G238S), then the lacZ alpha gene would be acquired and the zeocin resistance gene would be lost. Additionally, based on the reverse transcriptase primer and donor template used for this experiment, if the crossover occurs between the amino acid mutations of the two b-lactamase genes, recombinants should have the G238S and E104K mutations.

TABLE 1

| Sample ID | Donor Template | Recipient Template | Zeo/lacZ | E104K | G238S | CAM | Comments |
|---|---|---|---|---|---|---|---|
| Amp-RT/dsDNA-lac #1 | pCAZ/G238S | pKAL/E104K | ND | ND | yes | yes | |
| Amp-RT/dsDNA-lac #2 | pCAZ/G23BS | pKAL/E104K | lacZ | no | yes | yes | Recombinant |
| Amp-RT/dsDNA-lac #3 | pCAZ/G238S | pKAL/E104K | lacZ | yes | yes | yes | Recombinant |
| Amp-RT/dsDNA-lac #4 | pCAZ/G238S | pKAL/E104K | lacZ | yes | no | no | truncated due to |
| | | | | | | | non-specific priming |
| Amp-RT/dsDNA-lac #5 | pCAZ/G238S | pKAL/E104K | lacZ | no | yes | yes | Recombinant |
| Amp-RT/dsDNA-lac #6 | pCAZ/G238S | pKAL/E104K | lacZ | no | yes | yes | Recombinant |
| Amp-RT/dsDNA-lac #7 | pCAZ/G238S | pKAL/E104K | lacZ | no | yes | yes | Recombinant |
| Amp-RT/dsDNA-lac #8 | pCAZ/G238S | pKAL/E104K | lacZ | no | yes | yes | Recombinant |

ND = sequence not determined

### Example 2

Recombination Between Dehalogenase Variants

This example demonstrates how the RNA-dependent DNA polymerase MMLV reverse transcriptase can undergo template switching between homologous sequences at different crossover sites and is not limited to one crossover event. A dehalogenase gene variant with multiple restriction sites and amino acid mutations that differ from the wild-type sequence and the wild-type dehalogenase gene serve as the homologous sequences where template switching may occur by the RNA-dependent DNA polymerase MMLV reverse transcriptase.

Template Constructs

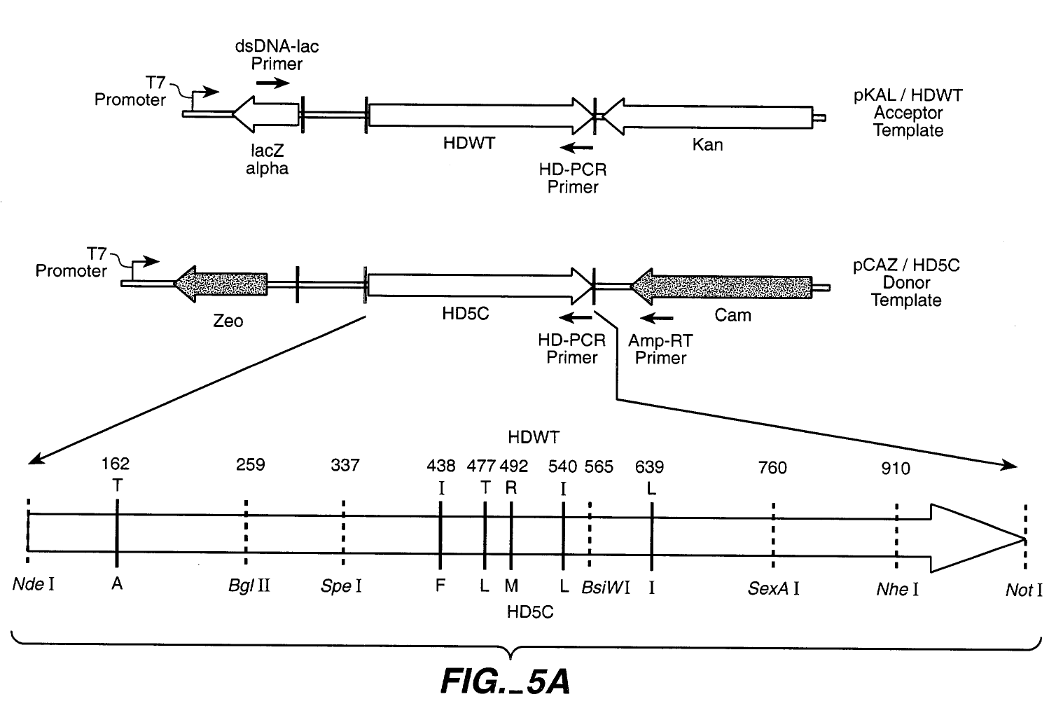

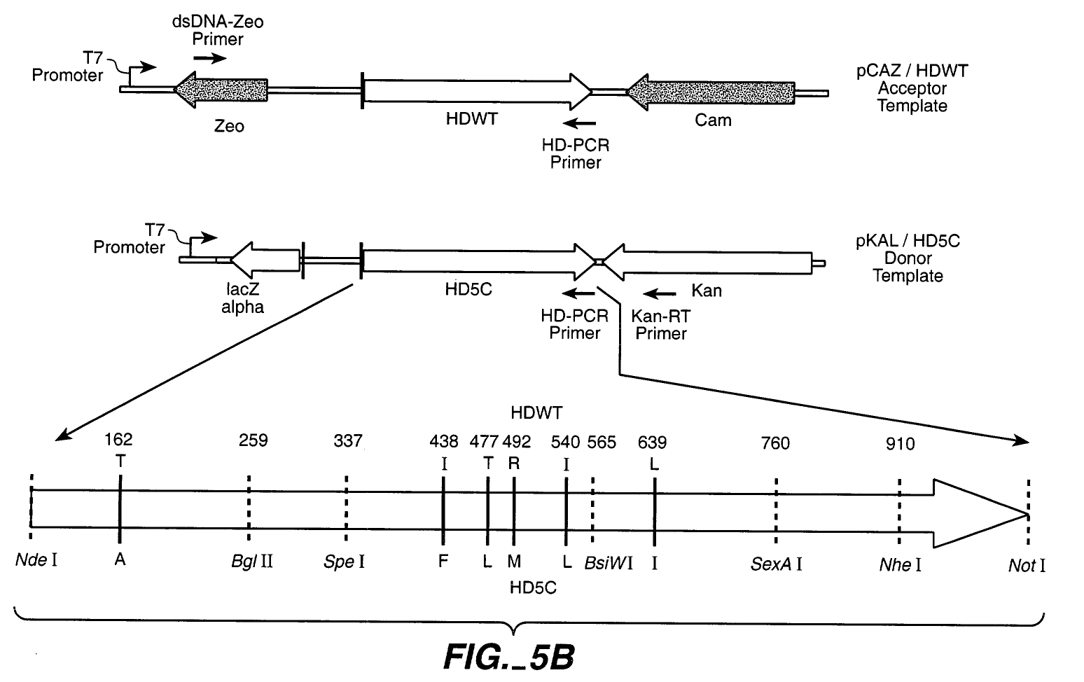

The dehalogenase genes were subcloned between the NdeI and NotI sites in place of the Amp gene of the pKAL and pCAZ vector constructs described in Example. 1. The different amino acid mutations and restriction sites between the two variants is shown in FIGS. 5A and B.

The procedure for template linearization and purification, in vitro transcription, reverse transcription and PCR are the same as in Example 1. The primer used for reverse transcription was Amp-RT if PCAZ/HD5C served as the donor template (FIG. 5A) or Kan-RT if pKAL/HD5C served as the donor template (FIG. 5B). The PCR primer pairs used for the reverse transcription reaction are shown in below in Table 2.

TABLE 2

| RT rxn | Donor Template | Recipient Template | RT-Primer | PCR primers |
|---|---|---|---|---|
| 1 | pCAZ/HD5 | pKAL/HDWT | Amp-RT (binds to CAM) | HD-PCR/dsDNA-lac |
| 2 | pKAL/HD5 | pCAZ/HDWT | Kan-RT (binds to KAN) | HD-PCR/Pst-dsDNA-Zeo |

PCR band products corresponding to the expected size were ligated into pCRBlunt 4 (Invitrogen) vector and transformed into Top 10 bacterial cells (Invitrogen). Transformation reactions corresponding to the Rxn 1 conditions (see Table 2) were plated on kanamycin and X-gal containing LB/agar plates. Blue colonies were randomly picked for sequencing. Transformation reactions corresponding to the Rxn 2 (see Table 2) were plated on zeocin containing LB/agar plates. Colonies were randomly picked for sequencing.

Sequencing Results and Analysis

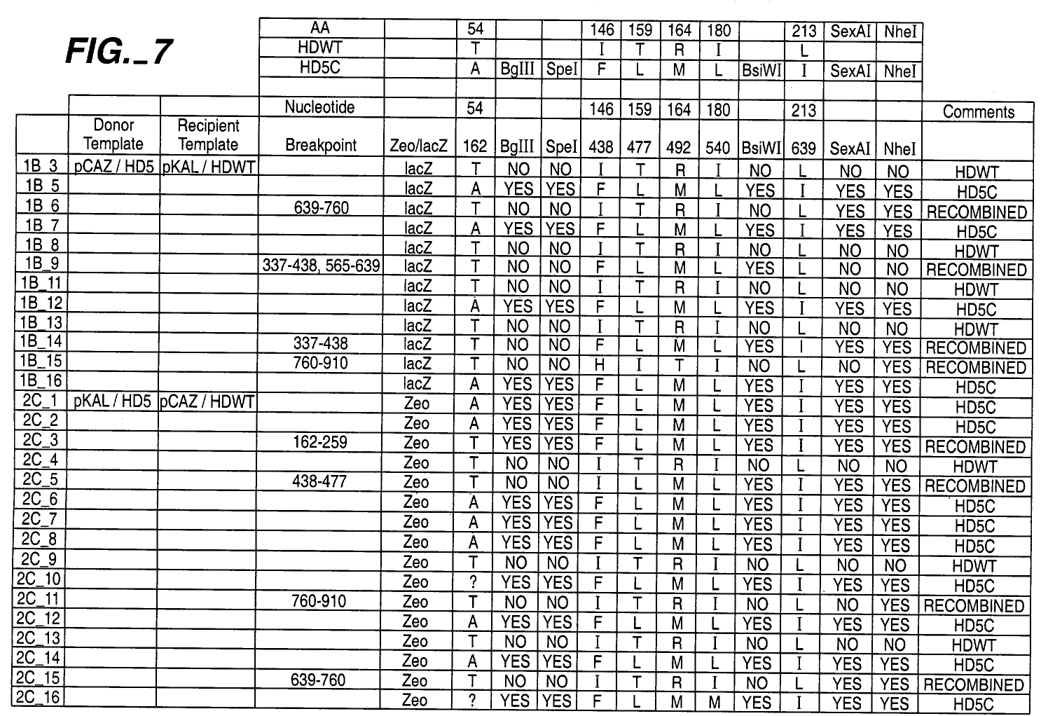

The sequencing results are shown in FIG. 7. 1B sequenced clones correspond to the Rxn1 conditions and 2C sequenced clones correspond to the Rxn2 conditions. The crossover regions can be distinguished for the clones labeled recombinant in the comments column. For the other clones, the gene markers that indicate recombination (i.e., zeo, lacZ) are present. Recombination can occur between the NheI and NotI for the clones labeled HDwt in the comments column or between the gene markers and amino acid position 54 for the clones labeled HD5C in the comments column. However, sequencing cannot distinguish where the crossover regions occur since the sequences are exactly identical between the two genes in these regions. The presence of zeo or lacZ gene would indicate RNA recombination has occurred.

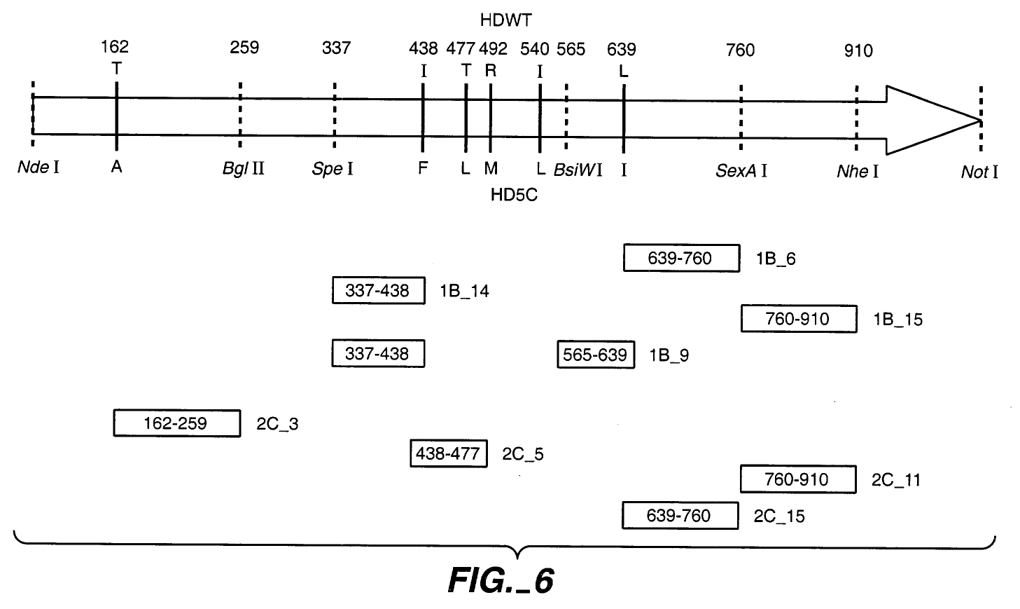

The sequencing results also indicate that recombination does not occur in one specific defined region but multiple regions as indicated in FIG. 6. Additionally, clone 1 B_{—}9 seems to have undergone three crossovers, the two indicated in FIG. 6 and between NheI and NotI since reverse transcription started with the HD5D gene; the NheI HD5D marker has been lost.

We claim:

1. A method for generating protein libraries comprising:
a) providing at least a first positive template ribonucleic acid (RNA) comprising:
i) a 3′ RNA dependent RNA polymerase (RdRp) recognition signal;
ii) a target gene; and
b) adding an RdRp enzyme and ribonucleotides (NTPs) to generate a plurality of negative recombinant nucleic acid strands;
c) adding a RT enzyme and dNTPs to generate a plurality of positive recombinant nucleic acid strands;
d) amplifying said positive recombinant strands to form nucleic acid amplicons;
e) incorporating said amplicons into expression vectors to generate a library of variant expression vectors; and
f) transforming a plurality of said variant expression vectors into a plurality of host cells.

2. A method according to claim 1, further comprising screening said host cells for a desired phenotype.

3. A method according to claim 2, wherein said template comprises a purification tag and said method further comprises isolating variant proteins from said cells.

4. A method for generating protein libraries comprising:
a) providing at least a first positive template ribonucleic acid (RNA) comprising:
i) a 3′ RNA dependent RNA polymerase (RdRp) recognition signal required for synthesis of a negative-strand;
ii) a 5′ RdRp recognition signal required for synthesis of a positive-strand;
iii) a target gene; and
b) adding an RdRp enzyme and ribonucleotides (NTPs) to generate a plurality of negative and positive recombinant nucleic acid strands;
c) amplifying said negative and positive variant recombinant strands to form nucleic acid amplicons;
e) incorporating said amplicons into expression vectors to generate a library of variant expression vectors.

5. A method according to claim 4, further comprising transforming a plurality of said variant expression vectors into a plurality of host cells.

6. A method according to claim 5, further comprising screening said host cells for a desired phenotype.

7. A method according to claim 6, wherein said template comprises a purification tag and said method further comprises isolating variant proteins from said cells.

8. A method for generating protein libraries comprising:
a) providing a plurality of first positive template ribonucleic acid (RNA) each comprising a different target gene; and
b) adding a reverse transcriptase (RT) enzyme and deoxyribonucleotides (dNTPs) to generate a plurality of negative and positive variant DNA recombinant strands;
c) amplifying said negative and positive recombinant strands to form amplicons;
d) incorporating said amplicons into expression vectors to generate a library of variant expression vectors; and,
e) transforming a plurality of said variant expression vectors into a plurality of host cells.

9. A method according to claim 8, further comprising screening said host cells for a desired phenotype.

10. A method according to claim 9, wherein said template comprises a purification tag and said method further comprises isolating variant proteins from said cells.

11. A method for generating protein libraries comprising:
a) providing at least one DNA template comprising:
i) a T7 promoter; and
ii) a target gene
b) providing a plurality of first positive template ribonucleic acid (RNA) each comprising a different target gene; and
c) adding a reverse transcriptase (RT) enzyme and deoxyribonucleotides (dNTPs) to generate a plurality of negative and positive DNA recombinant strands;
d) amplifying said negative and positive recombinant strands to form amplicons;
e) incorporating said amplicons into expression vectors to generate a library of variant expression vectors.; and,
f) transforming a plurality of said variant expression vectors into a plurality of host cells.

12. A method according to claim 11, further comprising screening said host cells for a desired phenotype.

13. A method according to claim 12, wherein said template comprises a purification tag and said method further comprises isolating variant proteins from said cells.

14. A method according to claim 1, 4, 8 or 11, further comprising:
d) synthesizing a plurality of said primary amplicon nucleic acid sequences; and
e) experimentally recombining said primary variant sequences to generate a secondary library comprising secondary variant sequences.

15. A method according to claim 1, 4, 8 or 11, further comprising:
d) synthesizing a plurality of said primary amplicon protein sequences; and
e) computationally recombining said primary variant sequences to generate a secondary library comprising secondary variant sequences.

16. A method according to claim 1, 4, 8 or 11, wherein said target gene is a naturally occurring gene.

17. A method according to claim 1, 4, 8 or 11, wherein said target gene is a designed gene.

18. A method according to claim 1, 4, 8 or 11, wherein said target genes are homologous genes. genes.

19. A method according to claim 1, 4, 8 or 11, wherein said target genes are non-homologous genes.

20. A method according to claim 1 or 4, wherein said RdRp is selected from the group consisting of cucumber mosaic cucumovirus, flock-house nodavirus, and toabacco mosaic tobamovirus.

21. A method according to claim 1 or 4, wherein said RdRp is a variant RdRp

22. A method according to claim 1 or 4 wherein said recognition signal comprises a hairpin motif.

23. A method according to claim 1 or 4 wherein said recognition signal comprises a A/U sequence.

24. A method for generating protein libraries comprising:
a) providing a host cell expressing an RdRp;
b) introducing at least a first template RNA into said host cell, said template comprising:
i) a 3′ RNA dependent RNA polymerase (RdRp) recognition signal required for synthesis of a negative-strand;
ii) a 5′ RdRp recognition signal required for synthesis of a positive-strand; and
iii) a target gene
c) generating a plurality of host cells containing different variant protein sequences; and
d) screening said host cells for a desired phenotype.

25. A method according to claim 24, wherein said template comprises a purification tag and said method further comprises isolating variant proteins from said cells.

26. A method for generating protein libraries comprising:
a) providing a host cell expressing an RdRp;
b) introducing at least a first template RNA into said host cell, said template comprising:
i) a 3′ RNA dependent RNA polymerase (RdRp) recognition signal required for synthesis of a negative-strand;
ii) a 5′ RdRp recognition signal required for synthesis of a positive-strand; and
iii) a target gene
c) generating a plurality of host cells comprising different variant nucleic acid sequences
d) amplifying said variant nucleic acid sequences;
e) incorporating said variant sequences into a library of expression vectors
f) transforming a plurality of cells with said expression vectors; and
g) screening said cells for a desired phenotype.

27. A method according to claim 26, wherein said template comprises a purification tag and said method further comprises isolating variant proteins from said cells.
